(19) United States
(12) Patent Application Publication
(10) Pub. No.: US 2024/0200042 A1
(43) Pub. Date: Jun. 20, 2024

(54) LIMITED SELF-REPLICATING MRNA MOLECULAR SYSTEM, PRODUCING METHOD AND USE

(71) Applicant: ZHENHE PHARMACEUTICAL (HANGZHOU) CO., LTD., HANGZHOU (CN)

(72) Inventors: GANG WANG, HANGZHOU (CN); YIN YU, HANGZHOU (CN); JIAN HUANG, HANGZHOU (CN); HUALIN YI, HANGZHOU (CN)

(21) Appl. No.: 18/598,120

(22) Filed: Mar. 7, 2024

Related U.S. Application Data
(63) Continuation of application No. PCT/CN2021/126076, filed on Oct. 25, 2021.

(30) Foreign Application Priority Data
Sep. 9, 2021 (CN) 202111054809.9
Sep. 15, 2021 (CN) 202111080355.2

(51) Int. Cl. C12N 9/12; A61P 9/00; C07K 14/005; C07K 14/435; C07K 14/47; C07K 14/55; C12N 9/22

(52) U.S. Cl. CPC C12N 9/127; A61P 9/00; C07K 14/005; C07K 14/435; C07K 14/43595; C07K 14/4715; C07K 14/55; C12N 9/22; C12Y 207/07048; C12N 2710/12022; C12N 2710/16622; C12N 2710/20022; C12N 2740/16022; C12N 2760/16122; C12N 2770/20022

(57) ABSTRACT

The application relates to the technical field of biomedicine, in particular to a limited self-replicating mRNA molecular system, producing method and use. The limited self-replicating mRNA molecular system including: a first mRNA encoding a mutated alphavirus replicase; and at least one second mRNA encoding a target protein; by generating specific mutation adjustments in the nsP2 subunit of mutated replicase, this limited self-replicating mRNA molecular system can achieve limited self-replication and avoid cytotoxicity; by constructing different mRNA with mutated alphavirus replicase and different target proteins, the mutated alphavirus replicase encoded by the first mRNA can simultaneously replicate multiple different target proteins, achieving sustained expression of multiple target proteins.

### CROSS-REFERENCE TO RELATED APPLICATIONS

This application is a continuation of International Application No. PCT/CN2021/126076 filed on Oct. 25, 2021, which claims priority to Chinese Patent Application No. 202111054809.9, filed on Sep. 9, 2021 and No. 202111080355.2, filed on Sep. 15, 2021, the entire contents of which are incorporated herein by reference.

### SEQUENCE LISTING

The instant application contains a Sequence Listing which has been submitted electronically in ST.26 format and is hereby incorporated by reference in its entirety. Said ST.26 copy, created on Mar. 6, 2024 is named SEQUENCE LISTING.xml and is 114 Kilobytes in size.

### FIELD OF THE DISCLOSURE

The application relates to the technical field of biomedicine, in particular to a limited self-replicating mRNA molecular system, producing method and use.

### BACKGROUND

Messenger RNA (mRNA) therapy is a novel treatment approach with broad clinical potential, including vaccines targeting infectious sources, treatments for cancer or genetic diseases, regenerative therapy, and immunotherapy. Compared to protein based biological agents, the advantages of messenger RNA therapy include that messenger RNA can synthesize proteins through the body's own cells, without the need for complex protein synthesis and purification processes or production lines; Intracellular and membrane binding proteins can be used as therapeutic targets; It can be rapidly industrialized under cell-free GMP conditions, with a short cycle from research and development to product development.

However, the application of messenger RNA therapy is limited by factors such as structural instability, innate immunogenicity, and inefficient in vivo delivery, the development direction of this technology lies in: Firstly, it must avoid rejection by the innate immune system, which may mistake therapeutic messenger RNA for non-self-nucleic acids, resulting in rejection. This is particularly important for repeated administration of messenger RNA therapy drugs, as immune memory may limit the effectiveness of drug products. Present research suggests that chemical modification of the nucleoside base of messenger RNA can reduce innate immune rejection and improve the efficiency of translating messenger RNA into proteins. However, it is still unclear how to carry out nucleoside modification, the proportion of modifications, and how to combine nucleotide modifications. Secondly, ordinary messenger RNA is unstable, easily degradable, and has a short expression duration. Studies have shown that ordinary messenger RNA can only be expressed in cells for 24 hours. Self-replicating messenger RNA, as it can self-replicate, can amplify the protein translation instructions of messenger RNA to enhance and prolong the expression of messenger RNA to proteins. The self-replicating messenger RNA molecular system used in existing technology originates from the genome skeleton of alphavirus, where the partial skeleton encoding viral RNA replicase is intact, and the skeleton encoding the virus structural protein is replaced by the target protein coding sequence. The messenger RNA molecular system has the following defects: firstly, compared to non-self-replicating messenger RNA, the nucleotide sequence of self-replicating messenger RNA is much longer, the cell burden is heavy, and in vitro transcription synthesis of messenger RNA is technically difficult, resulting in high industrial production costs; Secondly, the self-replicating messenger RNA molecular system is essentially an RNA pseudo virus that can self-replicate, and its viral properties are obvious, for example, it is impossible to predict the number of times it replicates, and there is a possibility of unlimited replication (pseudo virus reproduction in vivo), for another example, when vesicular stomatitis virus antigen and rabies virus antigen are packaged as self-replicating RNA mentioned above, there is a possibility of amplifying their toxicity. Thirdly, the messenger RNA molecular system mentioned above has high cytotoxicity, and due to the inability to be nucleoside modified, the immune response of cells or the body greatly exceeds that of non-self-replicating messenger RNA.

Therefore, it is necessary to provide a limited self-replicating messenger RNA molecular system.

### SUMMARY

The purpose of this application is to provide a limited self-replicating mRNA molecular system, producing method and use, to solve the technical problem of mRNA being unable to achieve limited self-replication in existing technologies.

The first aspect, the application provides a limited self-replicating mRNA molecular system, including:
a first mRNA encoding a mutated alphavirus replicase; and
at least one second mRNA encoding a target protein;
wherein the said mutated alphavirus replicase is mutated in the nsP2 region generating the mutant in position 259 and the mutant in position 650.

Optionally, the said mutated alphavirus replicase includes sequentially connected nsP1 region, nsP2 region, nsP3 region, and nsP4 region, the amino acid sequence of the said mutated alphavirus replicase is shown in SEQ ID NO: 1, the said mutated alphavirus replicase generated the mutant serine S to proline P in position 796 and the mutant arginine R to aspartate D in position 1187 of SEQ ID NO:1.

Optionally, the said first mRNA includes a mutated replicase coding sequence, the said mutated replicase coding sequence includes a RNA sequence corresponding to the nucleic acid sequence shown in SEQ ID NO.2;
each of the said second mRNA includes sequentially connected a 5′ end specific sequence for the replicase, a target protein coding sequence, and a 3′ end specific sequence for the replicase, the said 5′ end specific sequence is a RNA sequence corresponding to the nucleic acid sequence shown in SEQ ID NO.7, the said 3′ end specific sequence is a RNA sequence corresponding to the nucleic acid sequence shown in SEQ ID NO.8.

Optionally, any of the said first mRNA and the said second mRNA further includes a 5′ cap structure, a 5′UTR sequence, a 3′UTR sequence, and a polyadenylate sequence;
wherein the said first mRNA includes the following elements sequentially connected in the 5′ to 3′ direction: the 5′ cap structure, the 5′UTR sequence, the mutated replicase coding sequence, the 3′UTR sequence and the polyadenylate sequence;
each of the said second mRNA includes the following elements sequentially connected in the 5′ to 3′ direction: the 5′ cap structure, the 5′UTR sequence, the 5′ end specific sequence for the replicase, the target protein coding sequence, the 3′ end specific sequence for the replicase, the 3′UTR sequence and the polyadenylate sequence;

The said 5′UTR sequence includes a RNA sequence corresponding to the nucleic acid sequence shown in SEQ ID NO.9, the said 3′UTR sequence includes a RNA sequence corresponding to the nucleic acid sequence shown in SEQ ID NO.10, the said 5′ cap structure is selected from 3′-O-Me-m7G, m7GpppG, m2 7,3′-O GpppG, m7Gppp (5′) N1 and m7Gppp (m 2′-O) N1.

Optionally, part or all of the uracil in the said first mRNA or the said second mRNA has been chemically modified to enhance the stability of the first mRNA or second mRNA in the organism, the first mRNA or second mRNA has been chemically modified by replacing at least 50%, at least 60%, at least 70%, at least 80%, at least 90%, or 100% of uracil in the first mRNA or second mRNA with N1-methyl-pseudouridine.

Optionally, the said first mRNA and the said second mRNA are treated with RNase III, the said first mRNA and the said second mRNA are purified by fast protein liquid chromatography.

Optionally, the said target protein includes SARS-COV-2 antigenic peptides;
Or the said target protein includes IL-2 and Alpha-fetoprotein with no amino group;
Or the said target protein includes L1 protein of HPV6, L1 protein of HPV11, L1 protein of HPV16, L1 protein of HPV18, and E6 protein of HPV;
Or the said target protein includes envelope glycoprotein E of HSV and envelope glycoprotein D of HSV;
Or the said target protein includes Influenza virus HA antigen;
Or the said target protein includes HIV Gag antigen, HIV EnV antigen, and HIV CD40L;
Or the said target protein includes the NL-S protein of African swine fever virus, the cd2v ep402r protein of African swine fever virus, and the TK protein of African swine fever virus;
Or the said target protein includes Taffazin protein;
Or the said target protein includes C-Myc protein, Klf4 protein, Sox2 protein, OCT4 protein, and Lin28 protein;
Or the said target protein includes Cas9 protein and DNAJC19 protein;
Or the said target protein includes Hydrolyzed GFP protein.

The second aspect, the application provides a method for producing the limited self-replicating mRNA molecular system, including:
synthesize a first mRNA;
synthesize at least one second mRNA;
wherein the first mRNA encodes a mutated alphavirus replicase, each of the second mRNA encodes a target protein, the said mutated alphavirus replicase is mutated in the nsP2 region generating the mutant in position 259 and the mutant in position 650.

Optionally, also including:
treating the said first mRNA and the said second mRNA with RNase III;
purifying the said first mRNA and the said second mRNA by fast protein liquid chromatography.

Optionally, synthesize the first mRNA, including:
Synthesizing a mutated replicase DNA coding sequence, wherein the said mutated replicase DNA coding sequence includes a 5′UTR sequence shown in SEQ ID NO.9, a mutated replicase nucleic acid coding sequence shown in SEQ ID NO.2, and a 3′UTR sequence shown in SEQ ID NO.10;
Adding mRNA poly-(a) tail to the mutated replicase DNA coding sequence through PCR to obtain a DNA synthesis template for the first mRNA;
Synthesizing the first mRNA via transcription in vitro by the DNA synthesis template for the first mRNA.

Optionally, synthesize the second mRNA, including:
Synthesizing a specifically modified target protein DNA coding sequence, wherein the said specifically modified target protein DNA coding sequence includes a 5′UTR sequence shown in SEQ ID NO.9, a 5′ end specific sequence for the replicase shown in SEQ ID NO.7, a target protein DNA coding sequence, a 3′ end specific sequence for the replicase shown in SEQ ID NO.8 and a 3′UTR sequence shown in SEQ ID NO.10;
Adding mRNA poly-(a) tail to the specifically modified target protein DNA coding sequence through PCR to obtain a DNA synthesis template for the second mRNA;
Synthesizing the second mRNA via transcription in vitro by the DNA synthesis template for the second mRNA.

The third aspect, the application provides a biological material, the said biological material is any one of A1) to A6):
A1) a nucleic acid molecule encoding the first mRNA;
A2) a nucleic acid molecule encoding the second mRNA;
A3) a recombinant vector containing the nucleic acid molecule described in A1);
A4) a recombinant vector containing the nucleic acid molecule described in A2);
A5) transgenic animal cell lines containing the recombinant vector described in A3);
A6) transgenic animal cell lines containing the recombinant vector described in A4).

The fourth aspect, the application provides a drug combination, the said drug combination includes any mRNA molecular system, and a delivery agent.

The fifth aspect, the application provides a use of the first mRNA encoding a mutated alphavirus replicase as immunological adjuvant to regulate the immune system.

The sixth aspect, the application provides a use of any mRNA molecular system or biological material or drug combination in preparing cell re editing reagents, gene editing reagents, therapeutic drugs for Barth syndrome, vaccines for infectious diseases or tumor vaccines.

The beneficial effects of the present application are as follows: in this application the limited self-replicating mRNA molecular system including: a first mRNA encoding a mutated alphavirus replicase; and at least one second mRNA encoding a target protein; by generating specific mutation adjustments in the nsP2 subunit of mutated replicase, this limited self-replicating mRNA molecular system can achieve limited self-replication and avoid cytotoxicity; by constructing different mRNA with mutated alphavirus replicase and different target proteins, the mutated alphavirus replicase encoded by the first mRNA can simultaneously replicate multiple different target proteins, achieving sustained expression of multiple target proteins.

### BRIEF DESCRIPTION OF THE DRAWINGS

FIG. 1 shows the cardiac ejection fraction results in the Barth syndrome model mouse treatment experiment of the application;

FIG. 2A shows the staining diagram for cardiac pathological evaluation of induced wild type mice in the Barth syndrome model mouse treatment experiment of the application;

FIG. 2B shows the staining diagram for cardiac pathological evaluation of mice treated with common mRNA in the Barth syndrome model mouse treatment experiment of the application;

FIG. 2C shows the staining diagram for cardiac pathological evaluation of mice treated with bimolecular mRNA in the Barth syndrome model mouse treatment experiment of the application;

FIG. 3 shows the functional half-life and cellular innate immune rejection results of the limited self-replicating mRNA molecular system of the application;

FIG. 4 shows the results of low cytotoxicity effects of the limited self-replicating mRNA molecular system of the application;

FIG. 5 shows a results comparison of cell reprogramming of the limited self-replicating mRNA molecular system of the application;

FIG. 6A shows the staining results of cell reprogramming products of the limited self-replicating mRNA molecular system of the application;

FIG. 6B shows the staining results of cell reprogramming products of the limited self-replicating mRNA molecular system of the application;

FIG. 6C shows the staining results of cell reprogramming products of the limited self-replicating mRNA molecular system of the application;

FIG. 7A shows the result of DNAJC19 gene editing of the limited self-replicating mRNA molecular system of the application;

FIG. 7B shows the result of Taffazin gene editing of the limited self-replicating mRNA molecular system of the application;

FIG. 8 shows a schematic diagram of the structure of the limited self-replicating mRNA molecular system of the application.

### DETAILED DESCRIPTION OF PREFERRED EMBODIMENTS

The following will provide a clear and complete description of the technical solution in the embodiments of the invention, in conjunction with the accompanying drawings. Obviously, the described embodiments are only a part of the embodiments of the present application, not all of them. Based on the embodiments in this application, all other embodiments obtained by ordinary technical personnel in this field without creative labor fall within the scope of protection of this application.

Referring to “embodiments” in this article means that specific features, structures, or features described in conjunction with the embodiments may be included in at least one embodiment of the present application. The phrase appearing in various positions in the specification does not necessarily refer to the same embodiment, nor is it an independent or alternative embodiment that is mutually exclusive with other embodiments. Technicians in this field explicitly and implicitly understand that the embodiments described in this article can be combined with other embodiments.

The experimental methods in the following embodiments, unless otherwise specified, are all conventional methods. The materials, reagents, etc. used in the following embodiments can be obtained from commercial sources unless otherwise specified.

Positive stranded RNA virus genome serves as a template for translation and replication, it leads to multi-level interactions between host translation factors and RNA replication. All known positive stranded RNA viruses carry genes for RNA dependent RNA polymerase (RdRp) used for genome replication. However, unlike other RNA viruses, positive stranded RNA viruses do not shell the RNA polymerase. Therefore, when new cells are infected, viral RNA replication begins only when the genomic RNA is translated to produce RNA polymerase (which is also a replication factor for most positive stranded RNA viruses). All characterized positive stranded RNA viruses assemble their RNA replication complexes onto the cell membrane. Positive stranded RNA viruses produce negative stranded RNA, positive stranded RNA, double stranded RNA (dsRNA), and sub-genomic mRNA during replication, which themselves are strong inducers of innate immune response pathways.

The positive stranded RNA virus genome has the same polarity with cellular mRNA, and the positive stranded RNA virus genome RNA can be directly translated using the cellular translation system. Firstly, non-structural proteins are synthesized as precursor proteins and cleaved into mature non-structural proteins using viral proteases. Then, after translation and multi protein processing, a complex consisting of RNA polymerase (RdRp), additional non-structural proteins, viral RNA, and host cytokines is assembled. The assembled replication complex (RC) is used for viral RNA synthesis.

“RNA dependent RNA polymerase” or “RdRp” is known as an enzyme, protein, or peptide that catalyzes the de novo synthesis of RNA from RNA templates. A replicase is a complex of viral multi proteins or multi protein processing products that have RdRp activity and catalyze the replication of specific viral RNA. RdRp and the replicase are typically encoded by viruses with RNA genomes. Therefore, replicase not only provides the function of RNA dependent RNA polymerase, but also further includes additional viral non-structural multiprotein subunits that provide functions other than RdRp activity.

“Recombinant vector” refers to DNA or RNA based vector or plasmid that carries genetic information in the form of nucleic acid sequences. The terms “plasmid”, “vector”, “recombinant vector”, and/or “expression vector” may be used interchangeably in this article.

An example of the application provides a limited self-replicating mRNA molecular system, including: a first mRNA and at least one second mRNA; wherein the first mRNA encoding a mutated alphavirus replicase, each of the second mRNA encoding a target protein, limited replication of at least one target protein is achieved through mutated alphavirus replicase.

Wherein the said mutated alphavirus replicase is mutated in the nsP2 region generating the mutant in position 259 (serine S is mutated to proline P) and the mutant in position 650 (arginine R is mutated to aspartate D). Specifically, the mutated alphavirus replicase includes sequentially connected nsP1 region (537 amino acids), nsP2 region (799 amino acids), nsP3 region (482 amino acids), and nsP4 region (1254 amino acids), the amino acid sequence of the said mutated alphavirus replicase is shown in SEQ ID NO: 1, the mutated alphavirus replicase generated the mutant serine S to proline P in position 796 of SEQ ID NO:1 and the mutant arginine R to aspartate D in position 1187 of SEQ ID NO:1.

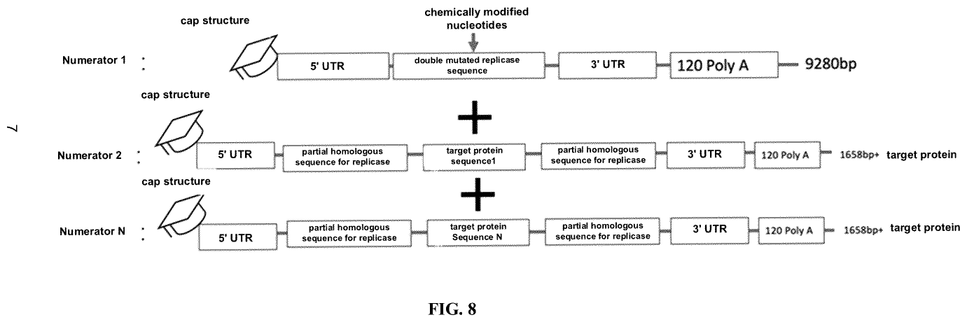

In this example, as shown in FIG. 8, including multiple second mRNA, the multiple second mRNA encode the first target protein, the second target protein, . . . , and the Nth target protein, respectively.

In an optional implementation, the first mRNA includes a mutated replicase coding sequence, the said mutated replicase coding sequence includes a RNA sequence corresponding to the nucleic acid sequence shown in SEQ ID NO.2. The nucleic acid sequence shown in SEQ ID NO.2 has high GC content, and the codon with high GC content is selected without changing the corresponding amino acid sequence, which is 7-20% higher than the GC content of the wild-type replicase DNA sequence. Specifically, as shown in SEQ ID NO.2, the high GC content DNA sequence at positions 1-1611 corresponds to the nsP1 region, the high GC content DNA sequence at positions 1612-4008 corresponds to the nsP2 region, the high GC content DNA sequence at positions 4009-5454 corresponds to the nsP3 region, the high GC content DNA sequence at positions 5455-9216 corresponds to the nsP4 region. The nucleic acid sequence shown in SEQ ID NO.11 is the replicase DNA sequence of wild alphavirus, where positions 1-1611 correspond to the original DNA sequence of nsP1 region, positions 1612-4008 correspond to the original DNA sequence of nsP2 region, positions 4009-5454 correspond to the original DNA sequence of nsP3 region, and positions 5455-9216 correspond to the original DNA sequence of nsP4 region.

In an optional implementation, each second mRNA includes sequentially connected a 5′ end specific sequence for the replicase, a target protein coding sequence, and a 3′ end specific sequence for the replicase, at both ends of the target protein coding sequence, specific sequences recognized of the alphavirus replicase are connected to improve the translation level of the target protein coding sequence, achieving the same effect without retaining the entire alphavirus RNA framework system, specifically, the 5′ end specific sequence is a RNA sequence corresponding to the nucleic acid sequence shown in SEQ ID NO.7, the 5′ end specific sequence is a RNA sequence is derived from the first to 221 st positions of the original DNA sequence corresponding to the nsP1 region of the replicase, the 3′ end specific sequence is a RNA sequence corresponding to the nucleic acid sequence shown in SEQ ID NO.8, the 3′ end specific sequence is derived from the second to 985th positions of the original DNA sequence corresponding to the nsP4 region of the replicase. The mRNA combination in this embodiment has limited replication, completely removing the virus attribute and completely eliminating the possibility of virus proliferation in vivo using the current alphavirus vector.

Furthermore, any of the said first mRNA and the said second mRNA further includes a 5′ cap structure, a 5′UTR sequence, a 3′UTR sequence, and a polyadenylate sequence; wherein the said first mRNA includes the following elements sequentially connected in the 5′ to 3′ direction: the 5′ cap structure, the 5′UTR sequence, the mutated replicase coding sequence, the 3′UTR sequence and the polyadenylate sequence. Similarly, each of the second mRNA includes the following elements sequentially connected in the 5′ to 3′ direction: the 5′ cap structure, the 5′UTR sequence, the 5′ end specific sequence for the replicase, the target protein coding sequence, the 3′ end specific sequence for the replicase, the 3′UTR sequence and the polyadenylate sequence. Specifically, the preferred target protein coding sequence is the RNA sequence corresponding to the open reading frame (ORF) in the target protein coding gene, the 5′UTR sequence includes a RNA sequence corresponding to the nucleic acid sequence shown in SEQ ID NO.9, the 3′UTR sequence includes a RNA sequence corresponding to the nucleic acid sequence shown in SEQ ID NO.10, the 5′ cap structure is selected from 3′-O-Me-m7G, m7GpppG, m2 7,3′-O GpppG, m7Gppp(5′)N1 and m7Gppp(m 2′-O)N1, preferably 3′-O-Me-m7G. The polyadenylate sequence is a sequence containing 60-200 adenylates; preferably, the polyadenylate sequence is a sequence containing 120 adenylates.

In the example, part or all of the uracil in the said first mRNA or the said second mRNA has been chemically modified to enhance the stability of the first mRNA or second mRNA in the organism, the first mRNA or second mRNA has been chemically modified by replacing at least 50%, at least 60%, at least 70%, at least 80%, at least 90%, or 100% of uracil in the first mRNA or second mRNA with N1-methyl-pseudouridine. Furthermore, in the example, 100% of uracil in the first mRNA or second mRNA has been replaced with N1-methyl-pseudouridine, to reduce innate immune rejection and to improve the efficiency of mRNA translation into proteins.

In the example, the first mRNA and second mRNA obtained by in vitro transcription of the recombinant vector are first treated with RNase III and then purified by fast protein liquid chromatography, which can further improve the efficiency of mRNA translation into protein.

In the example, theoretically, the target protein can be any acceptable protein or peptide, such as:

The limited self-replicating mRNA molecular system includes one second mRNA, encoding SARS-COV-2 antigenic peptides, the antigenic peptide can be selected from the receptor binding domain RBD of SARS-COV-2, the spike protein S1 subunit of SARS-COV-2, or the full-length sequence of spike protein S of SARS-COV-2; the above spike proteins are derived from SARS-COV-2 Delta mutant strains or SARS-COV-2 original strains.

The limited self-replicating mRNA molecular system includes two second mRNA, one encodes IL-2 and the other encodes Alpha-fetoprotein with no amino group.

The limited self-replicating mRNA molecular system includes five second mRNA, encode separately L1 protein of HPV6, L1 protein of HPV11, L1 protein of HPV16, L1 protein of HPV18, and E6 protein of HPV.

The limited self-replicating mRNA molecular system includes two second mRNA, encode separately envelope glycoprotein E of HSV and envelope glycoprotein D of HSV.

The limited self-replicating mRNA molecular system includes one second mRNA, encodes Influenza virus HA antigen.

The limited self-replicating mRNA molecular system includes five second mRNA, encode separately HIV Gag antigen, HIV EnV antigen, and HIV CD40L;
Or the said target protein includes the NL-S protein of African swine fever virus, the cd2v ep402r protein of African swine fever virus, and the TK protein of African swine fever virus;
The limited self-replicating mRNA molecular system includes one second mRNA, encodes Taffazin protein.

The limited self-replicating mRNA molecular system includes five second mRNA, encode separately C-Myc protein, Klf4 protein, Sox2 protein, OCT4 protein, and Lin28 protein.

The limited self-replicating mRNA molecular system includes two second mRNA, encode separately Cas9 protein and DNAJC19 protein;
Or the said target protein includes Hydrolyzed GFP protein.

An example of the application provides a biological material, the biological material including: (i) a nucleic acid molecule encoding the first mRNA; and (ii) a nucleic acid molecule encoding the second mRNA.

Wherein the nucleic acid molecule encoding the first mRNA include the nucleic acid sequence shown in SEQ ID NO.2, the nucleic acid molecule encoding the second mRNA include sequentially the nucleic acid sequence shown in SEQ ID NO.7, target protein DNA coding sequence, and the nucleic acid sequence shown in SEQ ID NO.8.

In an optional implementation, the nucleic acid molecule encoding the first mRNA include sequentially the nucleic acid sequence shown in SEQ ID NO.9, the nucleic acid sequence shown in SEQ ID NO.2, the nucleic acid sequence shown in SEQ ID NO.10, and polyadenylate sequence. The nucleic acid molecule encoding the second mRNA include sequentially the nucleic acid sequence shown in SEQ ID NO.9, the nucleic acid sequence shown in SEQ ID NO.7, target protein DNA coding sequence, the nucleic acid sequence shown in SEQ ID NO.8, the nucleic acid sequence shown in SEQ ID NO.10, and polyadenylate sequence.

An example of the application provides a biological material, the biological material including: a first recombinant vector containing the nucleic acid molecule encoding the first mRNA; and a second recombinant vector containing the nucleic acid molecules encoding the second mRNA.

An example of the application provides a biological material, the biological material including: a transgenic animal cell line containing the first recombinant vector; and a transgenic animal cell line containing the second recombinant vector.

### Example 1: Synthesis of the First mRNA

Step 1: Using GeneArt™ Gibson Assembly® HiFi reaction (Thermo Fisher, USA, A46624) synthesized the mutated replicase DNA coding sequence (the nucleic acid molecule encoding the first mRNA does not contain a polyadenylate sequence). After successful synthesis, the mutated replicase DNA coding sequence was cloned into the pcDNA3.3 vector plasmid for industrial production.

1.1 Mutant replicase DNA coding sequence: 5′UTR sequence (SEQ ID NO.9), mutant replicase coding sequence (SEQ ID NO.2), and 3′UTR sequence (SEQ ID NO.10). Among them, the mutant replicase coding sequence (SEQ ID NO.2) is divided into four DNA fragments: nsP1 region fragment (SEQ ID NO.3), nsP2 region fragment (SEQ ID NO.4), nsP3 region fragment (SEQ ID NO.5), and nsP4 region fragment (SEQ ID NO.6), All four DNA fragments are modified high GC content fragments. Four DNA fragments were directly ordered in gblock form from IDT Corporation in the United States.

The specific steps include assembling the Gibson reaction according to Table 1, reacting at 50° C. for 60 minutes in a PCR instrument to obtain the PCR product.

TABLE 1
Gibson reaction system

| Composition | dosage | volume |
|---|---|---|
| Gibson Carrier Kit | 0.08 pmol | 2 μL |
| Double mutated replicase fragment1(SEQ ID NO.3) | 0.08 pmol | 2 μL |
| Double mutated replicase fragment2(SEQ ID NO.4) | 0.08 pmol | 2 μL |
| Double mutated replicase fragment3(SEQ ID NO.5) | 0.08 pmol | 2 μL |
| Double mutated replicase fragment4(SEQ ID NO.6) | 0.08 pmol | 2 μL |
| GeneArt ™Gibson Assembly ® HF Master Mix | 2× | 10 μL |
| total | | 20 μL |

1.2 Transform PCR Products into One Shot™ TOP10 Chemically Responsive Escherichia coli Cells, Including the Following Steps:
Dilute the above Gibson reaction system (PCR product) with nuclease free water at a ratio of 1:5, 12 μL of nuclease free water and 3 μL of Gibson reaction system, mix well, react on ice;
Add 1 μL dilution solution to One Shot™ TOP10 chemically responsive Escherichia coli cells and mix, incubate the transformed mixture on ice for 20-30 minutes;
Incubate the cells at 42° C. for 30 seconds without shaking them;
Immediately transfer the reaction tube to ice and incubate on ice for 2 minutes;
Join 450 μL SOC culture medium in room temperature (Life Technology, USA);
Shake at 300 rpm for 1 hour at 37° C.;
Take 100 μL, apply to bacterial culture plate (100 μg/mL ampicillin or 50 μg/mL kanamycin);
Overnight at 37° C., select bacterial clones, shake the bacteria at 37 º C, and perform first generation sequencing to select plasmids containing the correct sequence of double mutated replicase sequences.
   Step 2: Add the Poly-(a) Tail mRNA Through PCR to Obtain the DNA Synthesis Template for the First mRNA

Among them, the poly-(a) tail contains 120 adenylates.

Prepare PCR premix according to Table 2 (total volume 200u L, eight reactions with 25 μL for each);

TABLE 2
Composition of PCR Premix

| Composition | dosage | final concentration |
|---|---|---|
| Kapa PRC mix(2×) | 100 μL | 1× |
| tailing primer-F1 10 μm(SEQ ID NO.12) | 6 μL | 0.3 uM |
| tailing primer-T120 10 μm(SEQ ID NO.13) | 6 μL | 0.3 uM |
| water | 80 μL | |
| Double mutant replicase linearized | 8 μL | 40-400 pg/μL |
| plasmid 10 ug/μL | | |

PCR reactions were performed according to the reaction conditions shown in Table 3;

TABLE 3
PCR reaction conditions

| cycle index | Denaturation | Annealing | Extension |
|---|---|---|---|
| 1 | 95° C., 2-3 min | | |
| 2-31 | 98° C., 20 s | 60° C., 15 s | 72° C., 60 s |
| 32 | 72° C., 3 min | | |

The quality of PCR products was checked by gel electrophoresis;
The PCR products were recovered by cutting the gel (QIAquick PCR purification kit, Qiagen, cat. no. 28106), and the final concentration of the tail template was adjusted to 100 ng/μL. As a DNA synthesis template for the first mRNA in vitro transcription.
   Step 3: In Vitro Transcription for Synthesis of the First mRNA

1. Assemble the mRNA cap structure and nucleotide mixture according to table 4:

Cap structure 3′-O-Me-m7G (5′) ppp (5′) G RNA cap analog (New England Biolabs, cat. no. S1411S), Methylcytidine-5′-triphosphate (Me-CTP; Trilink, cat. no. N1014),N1-methyl-pseudo-UTP (Trilink, cat. no. N1019), other components were from megascript T7 Kit (Ambion, cat. no. AM1334).

TABLE 4
mRNA cap structure and nucleotide mixture

| | Stock | | |
|---|---|---|---|
| | solution | | final |
| | con- | Volume of | con- |
| | centration | each IVT | centration |
| Composition | (mM) | reaction(ml) | (mM) |
| 3′-O-Me-m7G cap analog (NEB) | 60 | 4.0 | 6.0 |
| GTP (from MEGAscript T7 kit) | 75 | 0.8 | 1.5 |
| ATP (from MEGAscript T7 kit) | 75 | 4.0 | 7.5 |
| Me-CTP (from Trilink) | 100 | 3.0 | 7.5 |
| N1-methyl-pseudo-UTP (from Trilink) | 100 | 3.0 | 7.5 |

2. Assemble the in vitro transcription system of the first mRNA according to table 5:

TABLE 5
in vitro transcription system of the first mRNA

| | Dosage | final |
|---|---|---|
| Composition | (ml) | concentration |
| DNase/RNase-free water | 1.2 | |
| Custom NTP(from last step) | 14.8 | |
| Tailed PCR product, 100 ug/μL | 16 | 40 ng/μL |
| T7 buffer, 10×((from MEGAscript T7 kit) | 4.0 | 1× |
| T7 enzyme mix, 10× (from MEGAscript T7 kit) | 4.0 | 1× |

3. The reaction was incubated in the PCR instrument at 37° C. for 3˜6 h.

4. Add 2 μL turbo DNase (from MEGAscript T7 kit, Ambion, cat. No. am1334) to each sample.

5. Gently mix and incubate at 37 º C for 15 min.

6. The reactions treated with DNase and RNAa seIII were purified using MEGAclear Kit (Ambion, cat. No. am1908); eluted the modified mRNA with a total of 100 μL of elution buffer (50 μL of elution buffer eluted twice).

7. Purified modified mRNA was treated by phosphatase (Antarctic phosphatase (New England Biolabs, cat. No. m0289s)).

8. Add 11 μL 10× phosphatase buffer to each sample (about 100μ), followed by the addition of 2 μL phosphatase; gently mix the samples and then incubate them at 37° C. for 0.5˜1 h.

9. After elution, the concentration of the modified first mRNA was measured in a nanodrop spectrophotometer. The expected total production should be about 50 ug (range of 30˜70 ug; 100 μL elution volume for 40 μL of IVT response at a time was 300-700 ng/μL). The concentration was adjusted to 100 ng/μL by adding elution buffer or TE buffer (pH 7.0), or FPLC purification.

### Example 2: Synthesis of the Second mRNA

The synthesis steps of the second mRNA are similar to those of the first mRNA, including the following steps:

Step 1: Using GeneArt™ Gibson Assembly® HiFi reaction (Thermo Fisher, USA, A46624) synthesized the specifically modified target protein DNA coding sequence (the nucleic acid molecule encoding the second mRNA does not contain a polyadenylate sequence);

Wherein specifically modified target protein DNA coding sequence: 5′UTR sequence (SEQ ID No.9), 5′ end specific sequence for the replicase (SEQ ID No.7), target protein DNA coding sequence (shown in Table 6), 3′ end specific sequence for the replicase (SEQ ID No.8), 3′UTR sequence (SEQ ID No.10).

Step 2: Add the poly-(a) tail mRNA through PCR to obtain the DNA synthesis template for the second mRNA;

Step 3: in vitro transcription for synthesis of the second mRNA.

The 27 second mRNAs shown in Table 6 were synthesized according to the above methods.

TABLE 6
the DNA synthesis template for different second mRNA

| | | DNA sequence | specifically modified target protein DNA |
|---|---|---|---|
| | target protein | of target protein | coding sequence |
| second mRNA -1 | Taffazin protein | SEQ ID NO.14 | SEQ ID NO.9 + NO.7 + NO.14 + NO.8 + NO.10 |
| second mRNA -2 | Hydrolyzed GFP | SEQ ID NO.15 | SEQ ID NO.9 + NO.7 + NO.15 + NO.8 + NO.10 |
| | protein | | |
| second mRNA-3 | Cas9protein | SEQ ID NO.16 | SEQ ID NO.9 + NO.7 + NO.16 + NO.8 + NO.10 |
| second mRNA-4 | DNAJC19protein | SEQ ID NO.17 | SEQ ID NO.9 + NO.7 + NO.17 + NO.8 + NO.10 |
| second mRNA-5 | SARS-COV-2 | SEQ ID NO.18 | SEQ ID NO.9 + NO.7 + NO.18 + NO.8 + NO.10 |
| | antigenic peptides | | |
| | (wild spike protein S) | | |
| second mRNA-28 | SARS-COV-2 | SEQ ID NO.48 | SEQ ID NO.9 + NO.7 + NO.48 + NO.8 + NO.10 |
| | antigenic peptides | | |
| | (δ Strain spike | | |
| | protein S) | | |
| second mRNA-6 | IL-2 | SEQ ID NO.19 | SEQ ID NO.9 + NO.7 + NO.19 + NO.8 + NO.10 |
| second mRNA-7 | Alpha-fetoprotein with | SEQ ID NO.20 | SEQ ID NO.9 + NO.7 + NO.20 + NO.8 + NO.10 |
| | no amino group | | |
| second mRNA-8 | HPV6-L1protein | SEQ ID NO.21 | SEQ ID NO.9 + NO.7 + NO.21 + NO.8 + NO.10 |
| second mRNA-9 | HPV11-L1protein | SEQ ID NO.22 | SEQ ID NO.9 + NO.7 + NO.22 + NO.8 + NO.10 |
| second mRNA-10 | HPV16-L1protein | SEQ ID NO.23 | SEQ ID NO.9 + NO.7 + NO.23 + NO.8 + NO.10 |
| second mRNA-11 | HPV18-L1protein | SEQ ID NO.24 | SEQ ID NO.9 + NO.7 + NO.24 + NO.8 + NO.10 |
| second mRNA-12 | HPV-E6protein | SEQ ID NO.25 | SEQ ID NO.9 + NO.7 + NO.25 + NO.8 + NO.10 |
| second mRNA-13 | envelope glycoprotein | SEQ ID NO.26 | SEQ ID NO.9 + NO.7 + NO.26 + NO.8 + NO.10 |
| | E of HSV | | |
| second mRNA-14 | envelope glycoprotein | SEQ ID NO.27 | SEQ ID NO.9 + NO.7 + NO.27 + NO.8 + NO.10 |
| | D of HSV | | |
| second mRNA-15 | Influenza virus HA | SEQ ID NO.28 | SEQ ID NO.9 + NO.7 + NO.28 + NO.8 + NO.10 |
| | antigen | | |
| second mRNA-16 | HIV Gag antigen | SEQ ID NO.29 | SEQ ID NO.9 + NO.7 + NO.29 + NO.8 + NO.10 |
| second mRNA-17 | HIV EnV antigen | SEQ ID NO.30 | SEQ ID NO.9 + NO.7 + NO.30 + NO.8 + NO.10 |
| second mRNA-18 | HIV-CD40L | SEQ ID NO.31 | SEQ ID NO.9 + NO.7 + NO.31 + NO.8 + NO.10 |
| second mRNA-19 | NL-S protein of | SEQ ID NO.32 | SEQ ID NO.9 + NO.7 + NO.32 + NO.8 + NO.10 |
| | African swine fever | | |
| | virus | | |
| second mRNA-20 | cd2v ep402r protein | SEQ ID NO.33 | SEQ ID NO.9 + NO.7 + NO.33 + NO.8 + NO.10 |
| | of African swine fever | | |
| | virus | | |
| second mRNA-21 | TK (A240L)protein | SEQ ID NO.34 | SEQ ID NO.9 + NO.7 + NO.34 + NO.8 + NO.10 |
| | of African swine fever | | |
| | virus | | |
| second mRNA-22 | c-Myc protein | SEQ ID NO.35 | SEQ ID NO.9 + NO.7 + NO.35 + NO.8 + NO.10 |
| second mRNA-23 | Klf4 protein | SEQ ID NO.36 | SEQ ID NO.9 + NO.7 + NO.36 + NO.8 + NO.10 |
| second mRNA-24 | Sox2 protein | SEQ ID NO.37 | SEQ ID NO.9 + NO.7 + NO.37 + NO.8 + NO.10 |
| second mRNA-25 | OCT4 protein | SEQ ID NO.38 | SEQ ID NO.9 + NO.7 + NO.38 + NO.8 + NO.10 |
| second mRNA-26 | Lin28 protein | SEQ ID NO.39 | SEQ ID NO.9 + NO.7 + NO.39 + NO.8 + NO.10 |
| second mRNA-27 | rabies virus antigen | SEQ ID NO.47 | SEQ ID NO.9 + NO.7 + NO.47 + NO.8 + NO.10 |

The above SEQ ID No.14 to SEQ ID No.39 and SEQ ID No.47 were modified with high GC content on the basis of the corresponding original sequence without changing the original amino acid sequence.

### Example 3

An example of the application provides a drug combination, the drug combination includes multiplexed molecular messenger RNA and a delivery agent, wherein multiplexed molecular messenger RNA includes the first mRNA prepared in example 1 and the second mRNA-1 prepared in example 2.

### Application of Example 3: Barth Syndrome Model Mouse Treatment Experiment

### 3.1 Barth Syndrome Model Mouse and Inducing

Doxycycline was introduced into the mouse genome to induce the knock down of taffazin protein, and Barth syndrome model mouse was established. The genotyping DNA was determined by PCR analysis, primers were as follows:

(SEQ ID NO. 45)
5′CCATGGAATTCGAACGCTGACGTC 3′;
(SEQ ID NO. 46)
3′TATGGGCTATGAACTAATGACCC 5′;

In this case, only males were used. Doxycycline was placed in the drinking water of mice at a concentration of 2 mg/l, and 10% sucrose was also contained.

### 3.2 Multiple Molecular Messenger RNA Therapy:

10 μL protamine (protamine ipex5000, MEDA pharmaceutical company, 5000 IU/m) was added to 280 μL water, as 280 μL water+10 μL protamine 5000, prepare 0.5 mg/ml protamine solution, and 0.5 mg/ml multiple molecular messenger RNA (the molar ratio of multiple molecules is 1:1 in solution). Add an equal amount of protamine solution to the RNA solution, and quickly purge it up and down for at least 10 times. Leave it at room temperature for 10 minutes to prepare 130 nm protamine RNA nanoparticles, which are placed in the mouse subcutaneous pump (Alzet pump, https://www.alzet.com/guide-to-use/scid/) for continuous dosing.

Barth syndrome mice (TG) were divided into six groups: TG1, TG2, TG3, TG4, tg5, TG6;
Step 1: TG1, TG2, TG3, TG4, tg5, TG6 were induced by doxycycline for 8 weeks, and the cardiac ejection fraction FS % was detected;
Step 2: TG1, TG2, TG3, TG4, tg5, TG6 were induced by doxycycline for 10 weeks (continue to induce on the basis of step 1), and the cardiac ejection fraction FS % was detected;
Step 3: treating TG1, TG2, TG3, and TG4 with the pharmaceutical composition of example 3 for 2 weeks, while TG5 and TG6 have no treatment, and then the cardiac ejection fraction FS % was detected;
Step 4: when treating TG1, TG2, TG3, and TG4 with the pharmaceutical composition of example 3 for 3 weeks (continue the treatment for 1 week on the basis of step 3), while TG5 and TG6 have no treatment, and then the cardiac ejection fraction FS % was detected;
Step 5: when treating TG1, TG2, TG3, and TG4 with the pharmaceutical composition of example 3 for 6 weeks (continue the treatment for 3 weeks on the basis of step 4), while TG5 and TG6 have no treatment, and then the cardiac ejection fraction FS % was detected.

### 3.3 Evaluation of Forced Exercise Ability of Mice:

The mouse was operated on a closed electric treadmill with adjustable speed and inclination (slope angle) and also with an electric shock transmission network of an electric shock intensity of 1 mA. Initially, the animals were allowed to adapt to the environment by resting on the treadmill for 30 min, and the test started with a 10% slope and a speed of 5 m/min. Gradually increase 5 m/min every 5 minutes to a final speed of 25m/min.

### 3.4 Cardiac Function Evaluation by Ultrasound and Cardiac Fibrosis Evaluation by Sirius Red Staining of Barth Model Mice

### Grouping:

Group A: wild type mice were induced with doxycycline for 8 weeks;
Group B: Barth syndrome mice (TG) were induced with doxycycline for 8 weeks, and treated with common drug composition (common messenger RNA system+delivery system) for 6 weeks, in which the common messenger RNA system encoded taffazin protein; the common messenger RNA system is prepared according to the method recorded in the prior art CN201910014953.6;
Group C: Barth syndrome mice (TG) were induced with doxycycline for 8 weeks and treated with the pharmaceutical composition of example 3 for 6 weeks;
Cardiac function evaluation by ultrasound and cardiac fibrosis evaluation by Sirius red staining were taken in Group A, group B and group C respectively.

### 3.5 Experimental Results and Analysis:

### 3.5.1 Test Results of Cardiac Function Indicators

After multiple molecular messenger RNA therapy for Barth syndrome, cardiac ejection fraction ultrasound results suggest that multiple molecular messenger RNA therapy for Barth syndrome can improve its cardiac function.

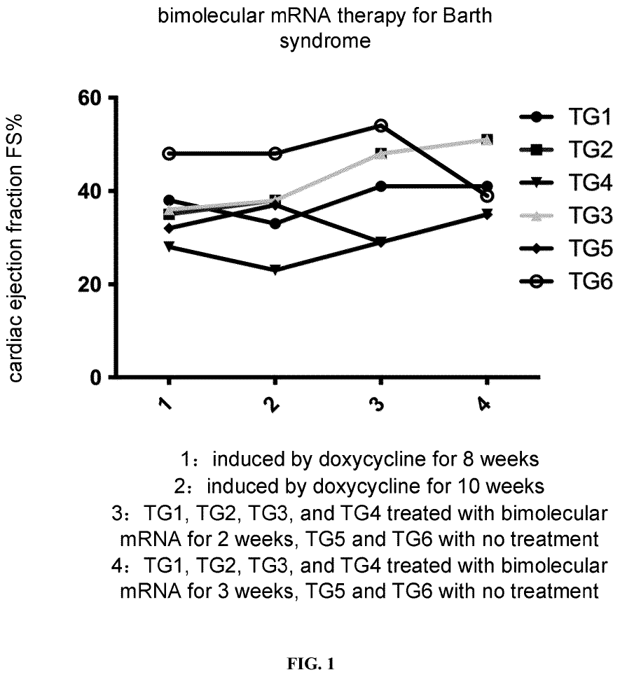

As shown in FIG. 1, the treatment of taffazin protein encoded by the limited replication multiplex molecular messenger RNA system improves the cardiac function of mice with congenital cardiomyopathy Barth syndrome. Specifically, the function of taffazin protein in mice with Barth syndrome (TG) is lost under doxycycline induction, the symptoms of Barth syndrome appear cardiomyopathy phenotype, the cardiac function index ejection fraction decreases, and the cardiac function of TG5 and TG6 without treatment decreases, in contrast, cardiac function of TG1, 2, 3, 4 improved after 2-3 weeks treatment with multiple molecular messenger RNA.

### 3.5.2 Test Results of Mice's Exercise Capacity

The forced exercise results of Barth syndrome treated with multiple molecular messenger RNA suggest that multiple molecular messenger RNA treatment of Barth syndrome can improve its exercise ability. The animals initially rested on the treadmill for 30 min to allow the animals to adapt to the environment, and the test started with a 10% slope and a speed of 5 m/min. Gradually increase 5 m/min every 5 minutes to the final speed of 25 m/min. Therefore, the duration of exercise was 36.8 minutes and the distance traveled was 507.4m. The results suggested that before the treatment, the model mice failed to maintain 15m/min and 10% inclination on the belt, and none of the model mice could maintain running when treadmill speed was more than 20 meters/minute, while after 6 weeks of multiple molecular messenger RNA treatment, the model mice could maintain running, indicating that multiple molecular messenger RNA treatment can significantly improve the exercise ability of the model mice.

### 3.5.3 Pathological Analysis Results

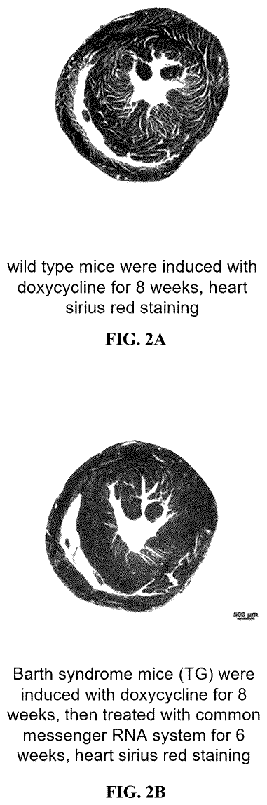

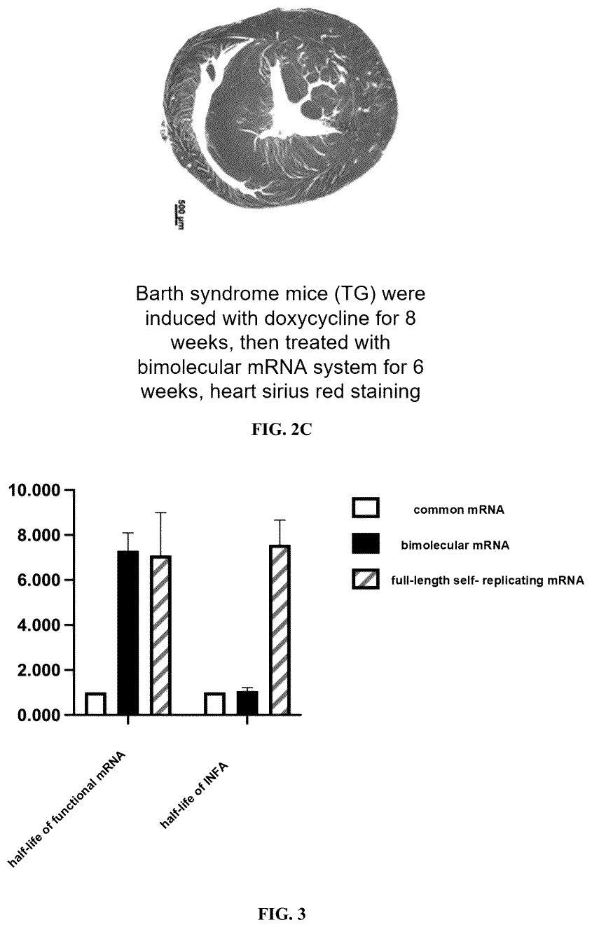

As shown in FIG. 2A to FIG. 2C, was the pathological analysis of Barth syndrome in congenital cardiomyopathy after the treatment of the limited replication multiplex messenger RNA molecular system by encoding taffazin protein.

The function of taffazin protein in Barth syndrome mice (TG) was lost under doxycycline induction. Barth syndrome symptoms appeared and cardiomyopathy phenotype appeared. Cardiac pathology suggested cardiac fibrosis. Multiple molecular messenger RNA treatment for 8 weeks significantly improved the degree of cardiac fibrosis, and was superior to the effect of common messenger RNA treatment.

### Example 4

An example of the application provides a limited self-replicating mRNA molecular system, the limited self-replicating mRNA molecular system includes the first mRNA prepared in example 1 and the second mRNA-2 prepared in example 2, the target protein is Hydrolyzed GFP protein.

### Application of Example 4

Cells were transfected with common mRNA encoding hydrolyzed GFP protein (Group 1), bimolecular mRNA from Example 4 (Group 2), and full-length self-replicating mRNA encoding hydrolyzed GFP (Group 3), respectively. The steps are shown in i-vii. the following expression of hydrolyzed GFP report gene (expressed GFP is rapidly degraded by its own hydrolytic enzyme, which can instantly reflect the duration and expression level of multiple messenger RNA molecules).

The steps for transfecting cells with a limited self-replicating mRNA molecular system in Example 4 are as follows:

(i) After thawing, 10 μL of the limited self-replicating mRNA molecular system from Example 4 (molar ratio of first mRNA and second mRNA-2 is 6:4) was added in 40 μL OPTI-MEM, then gently mix.

(ii) In another test tube, add 45 μL OPTI-MEM, and then add 5 μL Lipofectamine RNAiMax, then gently mix.

Repeat pipetting.

(iii) Add the diluted Lipofectamine RNAiMax to the diluted limited self-replicating mRNA molecular system of Example 4, and gently mix repeatedly.

(iv) Incubate the mixture at room temperature for 15 minutes.

(v) Using 100 μL the limited self-replicating mRNA molecular system/transfection reagent complex from Example 4 was uniformly added to one well of the six well plate.

(vi) Gently shake the board left and right to ensure the transfected complex can diffuse uniformly.

(vii) Put it to cell culture incubator with the 37° C., 5% CO2, and 5% O2.

By detecting the fluorescence intensity of GFP protein, the mRNA half-life in the cells of Group 1, the mRNA half-life in the cells of Group 2, and the mRNA half-life in the cells of Group 3 were detected, and cellular innate immune responses were performed on Group 1, Group 2, and third Group 3.

Detect the cells number after transfection in Group 1, Group 2, and third Group 3.

### Experimental Results and Analysis

As shown in FIG. 3, compared to full-length self-replicating messenger RNA, the half-life of limited self-replicating mRNA molecular system from Example 4 encoding the hydrolyzes GFP reporter gene does not show any difference. However, the cytotoxicity is weak, the immunogenicity is low, and compared to the common messenger RNA, the half-life of limited self-replicating mRNA molecular system from Example 4 is longer.

Please continue to refer to FIG. 3, the limited self-replicating mRNA molecular system from Example 4 has a long functional half-life and low cellular innate immune rejection. The half-life of the limited self-replicating mRNA molecular system from Example 4 is significantly higher than that of common messenger RNA, but similar to full-length self-replicating messenger RNA, but the cellular innate immune response (INFA, interferon A) is significantly lower than that of full-length self-replicating messenger RNA.

As shown in FIG. 4, the limited self-replicating mRNA molecular system (limited replicating multiple messenger RNA molecular system) from Example 4 exhibits low cytotoxicity. The cytotoxicity of the messenger RNA produced by the limited self-replicating mRNA molecular system from Example 4 is similar to that of ordinary messenger RNA, but significantly lower than that of full-length self-replicating messenger RNA.

### Example 5

An example of the application provides a limited self-replicating mRNA molecular system, the limited self-replicating mRNA molecular system includes the first mRNA prepared in example 1 and the second mRNA-22, the second mRNA-23, the second mRNA-24, the second mRNA-25, the second mRNA-26 prepared in example 2, the target proteins include C-Myc protein, Klf4 protein, Sox2 protein, OCT4 protein, and Lin28 protein.

### Application of Example 5: Cell Reprogramming Test

### Cell Reprogramming Steps:

1. Add 1 ml of 0.1% (wt/vol) gelatin to each well of the six well plate;

Leave at room temperature for at least 1 hour. Alternatively, overnight at 4 º C, one day before inoculating human NuFF feeder cells, remove gelatin by suction and let the plate dry at room temperature.

2. NuFF feeder cells (Newborn human foreskin fibroblasts, GlobalStem, cat. no. GSC-3001G), thaw a bottle of mitotic inactivated NuFFs and inoculate the cells onto a gelatin cell plate.

3. 6-12 hours after inoculating reprogrammed target fibroblasts, replace the fibroblast culture medium (Pluriton containing B18R (eBioscience, cat. no. 34-8185-85, 200 ng/ml)) with Pluriton (stemgent) completely reprogrammed medium, use 2 ml per well, and then incubate the cells overnight at 37° C., 5% CO2, and 5% 02 medium.

4. Use Lipofectamine RNAiMax (Invitrogen, cat. no. 56532) for transfection;

(i) After thawing, 10 μL of the limited self-replicating mRNA molecular system from Example 4 (molar ratio of double mutated replicase, OCT4, KLF4, c-MYC, SOX2, and LIN28A is 6:1:1:1:1:1 respectively, 100 ng/μL) was added in 40 μL OPTI-MEM, then gently mix.

(ii) In another test tube, add 45 μL OPTI-MEM, and then add 5 μL Lipofectamine RNAiMax, then gently mix.

Repeat pipetting.

(iii) Add the diluted Lipofectamine RNAiMax to the diluted limited self-replicating mRNA molecular system of Example 4, and gently mix repeatedly.

(iv) Incubate the mixture at room temperature for 15 minutes.

(v) Using 100 μL the limited self-replicating mRNA molecular system/transfection reagent complex from Example 4 was uniformly added to one well of the six well plate.

(vi) Gently shake the board left and right to ensure the transfected complex can diffuse uniformly.

(vii) Put it to cell culture incubator with the 37° C., 5% CO2, and 5% 02.

5. Repeat the i-vii steps every 72 hours until reprogrammed cell clones appear.

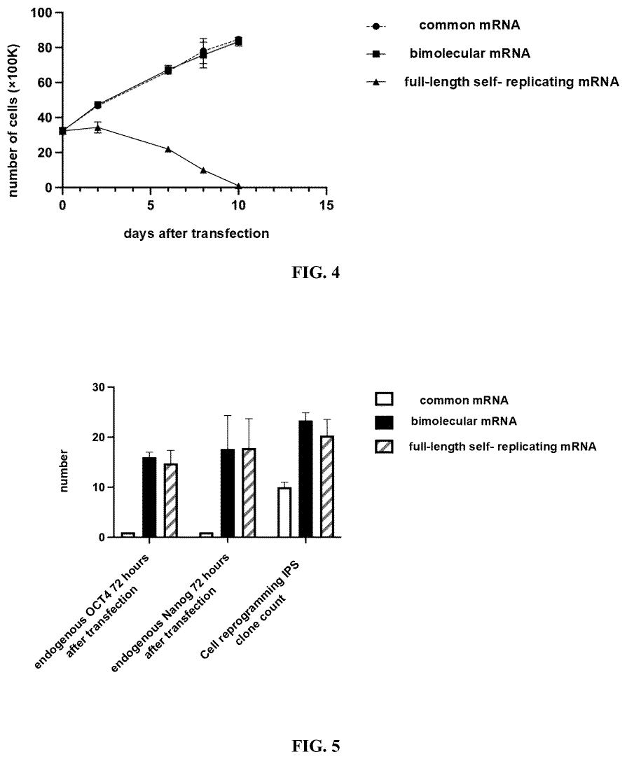

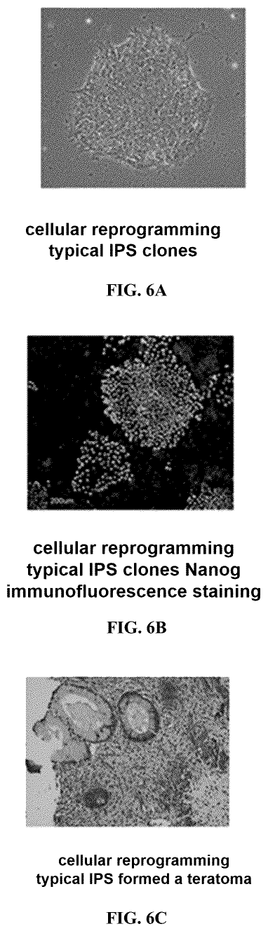

Please refer to FIGS. 5 and 6A, 6B, 6C, where the limited replication multiple messenger RNA molecular system simultaneously amplifies five encoding cell reprogramming factors Otc4, Sox2, Klf4, c-Myc, Lin28 (OSKML) to efficiently complete cell reprogramming; Compared to common messenger RNA, this system has longer protein expression and higher cell reprogramming (iPS clone count as an indicator); The iPS cells which were cellular reprogramming products produced by the limited self-replicating mRNA molecular system (limited replicating multiple messenger RNA molecular system) from Example 5 exhibit typical multipotency; The limited self-replicating mRNA molecular system (limited replicating multiple messenger RNA molecular system) from Example 5 encodes the 5 reprogramming factor OSKML to complete cell reprogramming, and after completing cell reprogramming, the product iPS cells display a classic multipotent stem cell clone appearance, and the multipotent marker Oct4 staining is positive, which can form teratomas in vivo.

### Example 6

An example of the application provides a limited self-replicating mRNA molecular system, the limited self-replicating mRNA molecular system includes the first mRNA prepared in example 1 and the second mRNA-3 prepared in example 2, the target protein is Cas9 protein.

### Application of Example 4: Gene Editing Experiment

### Implementation Steps:

The limited self-replicating mRNA molecular system (multiple messenger RNA) from Example 6 was subjected to DNA JC19 gene editing or Taffazin gene editing in induced pluripotent stem cells.

1. Electro-transfection of induced pluripotent stem cells: assemble the gene editing reaction system according to Table 7, including Taffazin gene gRNA sequence (SEQ ID NO.40, directly ordered from IDT company) and DNAJC19 gRNA sequence (SEQ ID NO.17, directly ordered from IDT company).

TABLE 7
Gene Editing Response System

| component | quality | volume |
|---|---|---|
| first mRNA (encoding double mutated replicase) | 2 ug | 2 μL |
| second mRNA-3 (encoding Cas9 protein) | 2 ug | 2 μL |
| Taffazin gene gRNA sequence or DNAJC19 | l ug | 2 μL |
| gRNA sequence | | |
| Electro-transfection buffer | | 14 μL |

Nucleofector 2b Device (Lonza, cat. no. AAB-1001), program B-016

| 2. Primers: | |
|---|---|
| (SEQ ID NO. 41) | |
| F: | TAAGCTAACCTGTCACCCCA; |
| (SEQ ID NO. 42) | |
| R: | AGAGCACAGAGGCGAGGCTT; |

PCR amplification of Taffazin gene fragments;

| Or primers: | |
|---|---|
| (SEQ ID NO. 43) | |
| F: | CTCAAAAGACTTCTGTTCTTGAGC; |
| (SEQ ID NO. 44) | |
| R: | CACTGAACACTGTGATAATCTGCT; |

PCR amplification of DNAJC19 gene fragment.

3. Surveyor enzyme evaluation of human induced stem cell Taffazin gene editing or DNAJC19 gene editing (IDT, cat. no. 706025), assembly of reaction system as shown in Table 8,

TABLE 8
Reaction System

| component | volume (μL) |
|---|---|
| 0.15 M MgCl₂ | 4 |
| Surveyor enhancer S | 1 |
| Surveyor nuclease S | 2 |

4. Mix evenly and incubate at 42° C. for 60 minutes.

5. Add 1/10 volume termination solution of the Surveyor Mutation Detection Kit to terminate the reaction and ⅙ volume of DNA.

6. The products of Surveyor nuclease digestion were analyzed by electrophoresis of 4-20% TBE gel at 200 V for about 60 minutes.

7. Dye the gel with 0.5 g/ml ethidium bromide in 1×TBE for 10 minutes. Wash the gel in water for 10 minutes.

8. The gel was imaged with an ultraviolet transmission apparatus.

### Experimental Results and Analysis

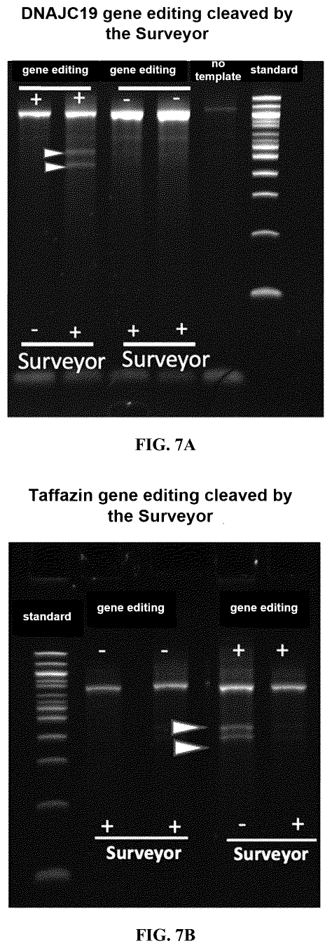

As shown in FIGS. 7A and 7B, the limited self-replicating mRNA molecular system (limited replicating multiple messenger RNA molecular system) of Example 6 encodes the CRISPR protein Cas9, efficiently editing the DNAJC19 and human Taffazin genes. Specifically, please refer to FIG. 7a, the DNAJC19 gene was successfully edited to produce a gene mutation, which was recognized and cleaved by the Surveyor, three typical bands appeared, indicating the completion of efficient gene editing. Please refer to FIG. 7B, the Taffazin gene was successfully edited to produce a gene mutation, which was recognized and cleaved by the Surveyor. Three typical bands appeared, indicating the completion of efficient gene editing.

### Example 7

The example provides a mRNA vaccine, includes the first mRNA prepared in example 1, the second mRNA-5 prepared in example 2, and protamine, form 130 nanometer protamine RNA particles for delivery. The target protein is SARS-COV-2 antigenic peptides (wild type spike protein S).

The example provides a mRNA vaccine, includes the first mRNA prepared in example 1, the second mRNA-28 prepared in example 2, and protamine, form 130 nanometer protamine RNA particles for delivery. The target protein is SARS-COV-2 antigenic peptides (Delta strain spike protein S).

### Example 8

The example provides a mRNA vaccine, includes the first mRNA prepared in example 1, the second mRNA-8, the second mRNA-9, the second mRNA-10, the second mRNA-11, the second mRNA-12 prepared in example 2, and protamine, form 130 nanometer protamine RNA particles for delivery. The target proteins include L1 protein of HPV6, L1 protein of HPV11, L1 protein of HPV16, L1 protein of HPV18, and E6 protein of HPV.

### Example 9

The example provides a mRNA vaccine, includes the first mRNA prepared in example 1, the second mRNA-13, and the second mRNA-14 prepared in example 2, and protamine, form 130 nanometer protamine RNA particles for delivery. The target proteins include envelope glycoprotein E of HSV and envelope glycoprotein D of HSV.

### Example 10

The example provides a mRNA vaccine, includes the first mRNA prepared in example 1, the second mRNA-15 prepared in example 2, and protamine, form 130 nanometer protamine RNA particles for delivery. The target protein is Influenza virus HA antigen.

### Example 11

The example provides a mRNA vaccine, includes the first mRNA prepared in example 1, the second mRNA-16, the second mRNA-17, and the second mRNA-18 prepared in example 2, and protamine, form 130 nanometer protamine RNA particles for delivery. The target proteins include HIV Gag antigen, HIV EnV antigen, and HIV CD40L.

### Example 12

The example provides a mRNA vaccine, includes the first mRNA prepared in example 1, the second mRNA-19, the second mRNA-20, and the second mRNA-21 prepared in example 2, and protamine, form 130 nanometer protamine RNA particles for delivery. The target proteins include the NL-S protein of African swine fever virus, the cd2v ep402r protein of African swine fever virus, and the TK protein of African swine fever virus.

### Example 13

The example provides a drug combination, used for treating colon cancer, includes the first mRNA prepared in example 1, the second mRNA-6, the second mRNA-7 prepared in example 2, and protamine, form 130 nanometer protamine RNA particles for delivery. The target proteins include IL-2 and Alpha-fetoprotein with no amino group.

### Example 14

The example provides a mRNA vaccine, includes the first mRNA prepared in example 1, the second mRNA-27 prepared in example 2, and protamine, form 130 nanometer protamine RNA particles for delivery. The target protein is rabies antigen (rabies glycoprotein).

The above is only the embodiment of the application. It should be pointed out here that ordinary technicians in the art can make improvements without departing from the creative idea of the application, but these belong to the protection scope of the application.

What is claimed is:

1. A limited self-replicating mRNA molecular system, comprising:
a first mRNA encoding a mutated alphavirus replicase; and
at least one second mRNA encoding a target protein;
wherein the said mutated alphavirus replicase is mutated in the nsP2 region generating the mutant in position 259 and the mutant in position 650.

2. The limited self-replicating mRNA molecular system of claim 1, wherein the said mutated alphavirus replicase comprises sequentially connected nsP1 region, nsP2 region, nsP3 region, and nsP4 region, the amino acid sequence of the said mutated alphavirus replicase is shown in SEQ ID NO:1, the said mutated alphavirus replicase generated the mutant serine S to proline P in position 796 and the mutant arginine R to aspartate D in position 1187 of SEQ ID NO:1.

3. The limited self-replicating mRNA molecular system of claim 1, wherein the said first mRNA comprises a mutated replicase coding sequence, the said mutated replicase coding sequence comprises a RNA sequence corresponding to the nucleic acid sequence shown in SEQ ID NO.2;
each of the said second mRNA comprises sequentially connected a 5′ end specific sequence for the replicase, a target protein coding sequence, and a 3′ end specific sequence for the replicase, the said 5′ end specific sequence is a RNA sequence corresponding to the nucleic acid sequence shown in SEQ ID NO.7, the said 3′ end specific sequence is a RNA sequence corresponding to the nucleic acid sequence shown in SEQ ID NO.8.

4. The limited self-replicating mRNA molecular system of claim 3, wherein any of the said first mRNA and the said second mRNA further comprises a 5′ cap structure, a 5′UTR sequence, a 3′UTR sequence, and a polyadenylate sequence;
wherein the said first mRNA comprises the following elements sequentially connected in the 5′ to 3′ direction: the 5′ cap structure, the 5′UTR sequence, the mutated replicase coding sequence, the 3′UTR sequence and the polyadenylate sequence;
each of the said second mRNA comprises the following elements sequentially connected in the 5′ to 3′ direction: the 5′ cap structure, the 5′UTR sequence, the 5′ end specific sequence for the replicase, the target protein coding sequence, the 3′ end specific sequence for the replicase, the 3′UTR sequence and the polyadenylate sequence;
The said 5′UTR sequence comprises a RNA sequence corresponding to the nucleic acid sequence shown in SEQ ID NO.9, the said 3′UTR sequence comprises a RNA sequence corresponding to the nucleic acid sequence shown in SEQ ID NO.10, the said 5′ cap structure is selected from 3′-O-Me-m7G, m7GpppG, m2 7,3′-O GpppG, m7Gppp(5′)N1 and m7Gppp(m 2′-O)N1.

5. The limited self-replicating mRNA molecular system of claim 1, wherein part or all of the uracil in the said first mRNA or the said second mRNA has been chemically modified to enhance the stability of the first mRNA or second mRNA in the organism, the first mRNA or second mRNA has been chemically modified by replacing at least 50%, at least 60%, at least 70%, at least 80%, at least 90%, or 100% of uracil in the first mRNA or second mRNA with N1-methyl-pseudouridine.

6. The limited self-replicating mRNA molecular system of claim 1, wherein the said first mRNA and the said second mRNA are treated with RNase III, the said first mRNA and the said second mRNA are purified by fast protein liquid chromatography.

7. The limited self-replicating mRNA molecular system of claim 1, wherein the said target protein comprises SARS-COV-2 antigenic peptides;
Or the said target protein comprises IL-2 and Alpha-fetoprotein with no amino group;
Or the said target protein comprises L1 protein of HPV6, L1 protein of HPV11, L1 protein of HPV16, L1 protein of HPV18, and E6 protein of HPV;
Or the said target protein comprises envelope glycoprotein E of HSV and envelope glycoprotein D of HSV;
Or the said target protein comprises Influenza virus HA antigen;
Or the said target protein comprises HIV Gag antigen, HIV EnV antigen, and HIV CD40L;
Or the said target protein comprises the NL-S protein of African swine fever virus, the cd2v ep402r protein of African swine fever virus, and the TK protein of African swine fever virus;
Or the said target protein comprises Taffazin protein;
Or the said target protein comprises C-Myc protein, Klf4 protein, Sox2 protein, OCT4 protein, and Lin28 protein;
Or the said target protein comprises Cas9 protein and DNAJC19 protein;
Or the said target protein comprises Hydrolyzed GFP protein.

8. A method for producing the limited self-replicating mRNA molecular system, comprising:
synthesize a first mRNA;
synthesize at least one second mRNA;
wherein the first mRNA encodes a mutated alphavirus replicase, each of the second mRNA encodes a target protein, the said mutated alphavirus replicase is mutated in the nsP2 region generating the mutant in position 259 and the mutant in position 650.

9. The method of claim 8, wherein also comprising:
treating the said first mRNA and the said second mRNA with RNase III;
purifying the said first mRNA and the said second mRNA by fast protein liquid chromatography.

10. The method of claim 8, wherein synthesize the first mRNA, comprising:
Synthesizing a mutated replicase DNA coding sequence, wherein the said mutated replicase DNA coding sequence comprises a 5′UTR sequence shown in SEQ ID NO.9, a mutated replicase nucleic acid coding sequence shown in SEQ ID NO.2, and a 3′UTR sequence shown in SEQ ID NO.10;
Adding mRNA poly-(a) tail to the mutated replicase DNA coding sequence through PCR to obtain a DNA synthesis template for the first mRNA;
Synthesizing the first mRNA via transcription in vitro by the DNA synthesis template for the first mRNA.

11. The method of claim 8, wherein synthesize the second mRNA, comprising:
Synthesizing a specifically modified target protein DNA coding sequence, wherein the said specifically modified target protein DNA coding sequence comprises a 5′UTR sequence shown in SEQ ID NO.9, a 5′ end specific sequence for the replicase shown in SEQ ID NO.7, a target protein DNA coding sequence, a 3′ end specific sequence for the replicase shown in SEQ ID NO.8 and a 3′UTR sequence shown in SEQ ID NO.10;
Adding mRNA poly-(a) tail to the specifically modified target protein DNA coding sequence through PCR to obtain a DNA synthesis template for the second mRNA;
Synthesizing the second mRNA via transcription in vitro by the DNA synthesis template for the second mRNA.

12. A biological material, the said biological material is any one of A1) to A6):
A1) a nucleic acid molecule encoding the first mRNA;
A2) a nucleic acid molecule encoding the second mRNA;
A3) a recombinant vector containing the nucleic acid molecule described in A1);
A4) a recombinant vector containing the nucleic acid molecule described in A2);
A5) transgenic animal cell lines containing the recombinant vector described in A3);
A6) transgenic animal cell lines containing the recombinant vector described in A4).

13. A drug combination, the said drug combination comprises the limited self-replicating mRNA molecular system of claim 1, and a delivery agent.

14. A use of the limited self-replicating mRNA molecular system of claim 1 in preparing cell re editing reagents, gene editing reagents, therapeutic drugs for Barth syndrome, vaccines for infectious diseases or tumor vaccines.


SEQUENCE LISTING (48 sequences)

SEQ ID NO: 1 - protein, 3071 aa, Synthetic construct
MAAKVHVDIEADSPFIKSLQKAFPSFEVESLQVTPNDHANARAFSHLATKLIEQETDKDTLILDIGSAPSRRMMSTHKYHCVCPMRSAEDPERLVCYAKKLAAASGKVLDREIAGKITDLQTVMATPDAESPTFCLHTDVTCRTAAEVAVYQDVYAVHAPTSLYHQAMKGVRTAYWIGFDTTPFMFDALAGAYPTYATNWADEQVLQARNIGLCAASLTEGRLGKLSILRKKQLKPCDTVMFSVGSTLYTESRKLLRSWHLPSVFHLKGKQSFTCRCDTIVSCEGYVVKKITMCPGLYGKTVGYAVTYHAEGFLVCKTTDTVKGERVSFPVCTYVPSTICDQMTGILATDVTPEDAQKLLVGLNQRIVVNGRTQRNTNTMKNYLLPIVAVAFSKWAREYKADLDDEKPLGVRERSLTCCCLWAFKTRKMHTMYKKPDTQTIVKVPSEFNSFVIPSLWSTGLAIPVRSRIKMLLAKKTKRELIPVLDASSARDAEQEEKERLEAELTREALPPLVPIAPAETGVVDVDVEELEYHAGAGVVETPRSALKVTAQPNDVLLGNYVVLSPQTVLKSSKLAPVHPLAEQVKIITHNGRAGRYQVDGYDGRVLLPCGSAIPVPEFQALSESATMVYNEREFVNRKLYHIAVHGPSLNTDEENYEKVRAERTDAEYVFDVDKKCCVKREEASGLVLVGELTNPPFHEFAYEGLKIRPSAPYKTTVVGVFGVPGSGKSAIIKSLVTKHDLVTSGKKENCQEIVNDVKKHRGLDIQAKTVDSILLNGCRRAVDILYVDEAFACHPGTLLALIALVKPRSKVVLCGDPKQCGFFNMMQLKVNFNHNICTEVCHKSISRRCTRPVTAIVSTLHYGGKMRTTNPCNKPIIIDTTGQTKPKPGDIVLTCFRGWVKQLQLDYRGHEVMTAAASQGLTRKGVYAVRQKVNENPLYAPASEHVNVLLTRTEDRLVWKTLAGDPWIKVLSNIPQGNFTATLEEWQEEHDKIMKVIEGPAAPVDAFQNKANVCWAKSLVPVLDTAGIRLTAEEWSTIITAFKEDRAYSPVVALNEICTKYYGVDLDSGLFSAPKVSLYYENNHWDNRPGGRMYGFNAATAARLEARHTFLKGQWHTGKQAVIAERKIQPLSVLDNVIPINRRLPHALVAEYKTVKGSRVEWLVNKVRGYHVLLVSEYNLALPRRDVTWLSPLNVTGADRCYDLSLGLPADAGRFDLVFVNIHTEFRIHHYQQCVDHAMKLQMLGGDALRLLKPGGSLLMRAYGYADKISEAVVSSLSRKFSSARVLRPDCVTSNTEVFLLFSNFDNGKRPSTLHQMNTKLSAVYAGEAMHTAGCAPSYRVKRADIATCTEAAVVNAANARGTVGDGVCRAVAKKWPSAFKGEATPVGTIKTVMCGSYPVIHAVAPNFSATTEAEGDRELAAVYRAVAAEVNRLSLSSVAIPLLSTGVFSGGRDRLQQSLNHLFTAMDATDADVTIYCRDKSWEKKIQEAIDMRTAVELLNDDVELTTDLVRVHPDSSLVGRKGYSTTDGSLYSYFEGTKFNQAAIDMAEILTLWPRLQEANEQICLYALGETMDNIRSKCPVNDSDSSTPPRTVPCLCRYAMTAERIARLRSHQVKSMVVCSSFPLPKYHVDGVQKVKCEKVLLFDPTVPSVVSPRKYAASTTDHSDRSLRGFDLDWTTDSSSTASDTMSLPSLQSCDIDSIYEPMAPIVVTADVHPEPAGIADLAADVHPEPADHVDLENPIPPPRPKRAAYLASRAAERPVPAPRKPTPAPRTAFRNKLPLTFGDFDEHEVDALASGITFGDFDDVLRLGRAGAMNYIPTQTFYGRRWRPRPAARPWPLQATPVAPVVPDFQAQQMQQLISAVNALTMRQNAIAPARPPKPKKKKTTKPKPKTQPKKINGKTQQQKKKDKQADKKKKKPGKRERMCMKIENDCIFEVKHEGKVTGYACLVGDKVMKPAHVKGVIDNADLAKLAFKKSSKYDLECAQIPVHMRSDASKYTHEKPEGHYNWHHGAVQYSGGRFTIPTGAGKPGDSGRPIFDNKGRVVAIVLGGANEGSRTALSVVTWNKDMVTRVTPEGSEEWSAPLITAMCVLANATFPCFQPPCVPCCYENNAEATLRMLEDNVDRPGYYDLLQAALTCRNGTRHRRSVSQHFNVYKATRPYIAYCADCGAGHSCHSPVAIEAVRSEATDGMLKIQFSAQIGIDKSDNHDYTKIRYADGHAIENAVRSSLKVATSGDCFVHGTMGHFILAKCPPGEFLQVSIQDTRNAVRACRIQYHHDPQPVGREKFTIRPHYGKEIPCTTYQQTTAKTVEEIDMHMPPDTPDRTLLSQQSGNVKITVGGKKVKYNCTCGTGNVGTTNSDMTINTCLIEQCHVSVTDHKKWQFNSPFVPRADEPARKGKVHIPFPLDNITCRVPMAREPTVIHGKREVTLHLHPDHPTLFSYRTLGEDPQYHEEWVTAAVERTIPVPVDGMEYHWGNNDPVRLWSQLTTEGKPHGWPHQIVQYYYGLYPAATVSAVVGMSLLALISIFASCYMLVAARSKCLTPYALTPGAAVPWTLGILCCAPRAHAASVAETMAYLWDQNQALFWLEFAAPVACILIITYCLRNVLCCCKSLSFLVLLSLGATARAYEHSTVMPNVVGFPYKAHIERPGYSPLTLQMQVVETSLEPTLNLEYITCEYKTVVPSPYVKCCGASECSTKEKPDYQCKVYTGVYPFMWGGAYCFCDSENTQLSEAYVDRSDVCRHDHASAYKAHTASLKAKVRVMYGNVNQTVDVYVNGDHAVTIGGTQFIFGPLSSAWTPFDNKIVVYKDEVFNQDFPPYGSGQPGRFGDIQSRTVESNDLYANTALKLARPSPGMVHVPYTQTPSGFKYWLKEKGTALNTKAPFGCQIKTNPVRAMNCAVGNIPVSMNLPDSAFTRIVEAPTIIDLTCTVATCTHSSDFGGVLTLTYKTDKNGDCSVHSHSNVATLQEATAKVKTAGKVTLHFSTASASPSFVVSLCSARATCSASCEPPKDHIVPYAASHSNVVFPDMSGTALSWVQKISGGLGAFAIGAILVLVVVTCIGLRR

SEQ ID NO: 2 - DNA, 9216 bp, Synthetic construct
  source 1..9216: mol_type = other DNA
atggccgccaaggtgcacgtggacatcgaggccgacagccccttcatcaagagcctgcagaaggccttccccagcttcgaggtggagagcctgcaggtgacccccaacgaccacgccaacgcccgggccttcagccacctggccaccaagctgatcgagcaggagaccgacaaggacaccctgatcctggacatcggcagcgcccccagccggcggatgatgagcacccacaagtaccactgcgtgtgccccatgcggagcgccgaggaccccgagcggctggtgtgctacgccaagaagctggccgccgccagcggcaaggtgctggaccgggagatcgccggcaagatcaccgacctgcagaccgtgatggccacccccgacgccgagagccccaccttctgcctgcacaccgacgtgacctgccggaccgccgccgaggtggccgtgtaccaggacgtgtacgccgtgcacgcccccaccagcctgtaccaccaggccatgaagggcgtgcggaccgcctactggatcggcttcgacaccacccccttcatgttcgacgccctggccggcgcctaccccacctacgccaccaactgggccgacgagcaggtgctgcaggcccggaacatcggcctgtgcgccgccagcctgaccgagggccggctgggcaagctgagcatcctgcggaagaagcagctgaagccctgcgacaccgtgatgttcagcgtgggcagcaccctgtacaccgagagccggaagctgctgcggagctggcacctgcccagcgtgttccacctgaagggcaagcagagcttcacctgccggtgcgacaccatcgtgagctgcgagggctacgtggtgaagaagatcaccatgtgccccggcctgtacggcaagaccgtgggctacgccgtgacctaccacgccgagggcttcctggtgtgcaagaccaccgacaccgtgaagggcgagcgggtgagcttccccgtgtgcacctacgtgcccagcaccatctgcgaccagatgaccggcatcctggccaccgacgtgacccccgaggacgcccagaagctgctggtgggcctgaaccagcggatcgtggtgaacggccggacccagcggaacaccaacaccatgaagaactacctgctgcccatcgtggccgtggccttcagcaagtgggcccgggagtacaaggccgacctggacgacgagaagcccctgggcgtgcgggagcggagcctgacctgctgctgcctgtgggccttcaagacccggaagatgcacaccatgtacaagaagcccgacacccagaccatcgtgaaggtgcccagcgagttcaacagcttcgtgatccccagcctgtggagcaccggcctggccatccccgtgcggagccggatcaagatgctgctggccaagaagaccaagcgggagctgatccccgtgctggacgccagcagcgcccgggacgccgagcaggaggagaaggagcggctggaggccgagctgacccgggaggccctgccccccctggtgcccatcgcccccgccgagaccggcgtggtggacgtggacgtggaggagctggagtaccacgccggcgccggcgtggtggagaccccccggagcgccctgaaggtgaccgcccagcccaacgacgtgctgctgggcaactacgtggtgctgagcccccagaccgtgctgaagagcagcaagctggcccccgtgcaccccctggccgagcaggtgaagatcatcacccacaacggccgggccggccggtaccaggtggacggctacgacggccgggtgctgctgccctgcggcagcgccatccccgtgcccgagttccaggccctgagcgagagcgccaccatggtgtacaacgagcgggagttcgtgaaccggaagctgtaccacatcgccgtgcacggccccagcctgaacaccgacgaggagaactacgagaaggtgcgggccgagcggaccgacgccgagtacgtgttcgacgtggacaagaagtgctgcgtgaagcgggaggaggccagcggcctggtgctggtgggcgagctgaccaacccccccttccacgagttcgcctacgagggcctgaagatccggcccagcgccccctacaagaccaccgtggtgggcgtgttcggcgtgcccggcagcggcaagagcgccatcatcaagagcctggtgaccaagcacgacctggtgaccagcggcaagaaggagaactgccaggagatcgtgaacgacgtgaagaagcaccggggcctggacatccaggccaagaccgtggacagcatcctgctgaacggctgccggcgggccgtggacatcctgtacgtggacgaggccttcgcctgccaccccggcaccctgctggccctgatcgccctggtgaagccccggagcaaggtggtgctgtgcggcgaccccaagcagtgcggcttcttcaacatgatgcagctgaaggtgaacttcaaccacaacatctgcaccgaggtgtgccacaagagcatcagccggcggtgcacccggcccgtgaccgccatcgtgagcaccctgcactacggcggcaagatgcggaccaccaacccctgcaacaagcccatcatcatcgacaccaccggccagaccaagcccaagcccggcgacatcgtgctgacctgcttccggggctgggtgaagcagctgcagctggactaccggggccacgaggtgatgaccgccgccgccagccagggcctgacccggaagggcgtgtacgccgtgcggcagaaggtgaacgagaaccccctgtacgcccccgccagcgagcacgtgaacgtgctgctgacccggaccgaggaccggctggtgtggaagaccctggccggcgacccctggatcaaggtgctgagcaacatcccccagggcaacttcaccgccaccctggaggagtggcaggaggagcacgacaagatcatgaaggtgatcgagggccccgccgcccccgtggacgccttccagaacaaggccaacgtgtgctgggccaagagcctggtgcccgtgctggacaccgccggcatccggctgaccgccgaggagtggagcaccatcatcaccgccttcaaggaggaccgggcctacagccccgtggtggccctgaacgagatctgcaccaagtactacggcgtggacctggacagcggcctgttcagcgcccccaaggtgagcctgtactacgagaacaaccactgggacaaccggcccggcggccggatgtacggcttcaacgccgccaccgccgcccggctggaggcccggcacaccttcctgaagggccagtggcacaccggcaagcaggccgtgatcgccgagcggaagatccagcccctgagcgtgctggacaacgtgatccccatcaaccggcggctgccccacgccctggtggccgagtacaagaccgtgaagggcagccgggtggagtggctggtgaacaaggtgcggggctaccacgtgctgctggtgagcgagtacaacctggccctgccccggcgggacgtgacctggctgagccccctgaacgtgaccggcgccgaccggtgctacgacctgagcctgggcctgcccgccgacgccggccggttcgacctggtgttcgtgaacatccacaccgagttccggatccaccactaccagcagtgcgtggaccacgccatgaagctgcagatgctgggcggcgacgccctgcggctgctgaagcccggcggcagcctgctgatgcgggcctacggctacgccgacaagatcagcgaggccgtggtgagcagcctgagccggaagttcagcagcgcccgggtgctgcggcccgactgcgtgaccagcaacaccgaggtgttcctgctgttcagcaacttcgacaacggcaagcggcccagcaccctgcaccagatgaacaccaagctgagcgccgtgtacgccggcgaggccatgcacaccgccggctgcgcccccagctaccgggtgaagcgggccgacatcgccacctgcaccgaggccgccgtggtgaacgccgccaacgcccggggcaccgtgggcgacggcgtgtgccgggccgtggccaagaagtggcccagcgccttcaagggcgaggccacccccgtgggcaccatcaagaccgtgatgtgcggcagctaccccgtgatccacgccgtggcccccaacttcagcgccaccaccgaggccgagggcgaccgggagctggccgccgtgtaccgggccgtggccgccgaggtgaaccggctgagcctgagcagcgtggccatccccctgctgagcaccggcgtgttcagcggcggccgggaccggctgcagcagagcctgaaccacctgttcaccgccatggacgccaccgacgccgacgtgaccatctactgccgggacaagagctgggagaagaagatccaggaggccatcgacatgcggaccgccgtggagctgctgaacgacgacgtggagctgaccaccgacctggtgcgggtgcaccccgacagcagcctggtgggccggaagggctacagcaccaccgacggcagcctgtacagctacttcgagggcaccaagttcaaccaggccgccatcgacatggccgagatcctgaccctgtggccccggctgcaggaggccaacgagcagatctgcctgtacgccctgggcgagaccatggacaacatccggagcaagtgccccgtgaacgacagcgacagcagcacccccccccggaccgtgccctgcctgtgccggtacgccatgaccgccgagcggatcgcccggctgcggagccaccaggtgaagagcatggtggtgtgcagcagcttccccctgcccaagtaccacgtggacggcgtgcagaaggtgaagtgcgagaaggtgctgctgttcgaccccaccgtgcccagcgtggtgagcccccggaagtacgccgccagcaccaccgaccacagcgaccggagcctgcggggcttcgacctggactggaccaccgacagcagcagcaccgccagcgacaccatgagcctgcccagcctgcagagctgcgacatcgacagcatctacgagcccatggcccccatcgtggtgaccgccgacgtgcaccccgagcccgccggcatcgccgacctggccgccgacgtgcaccccgagcccgccgaccacgtggacctggagaaccccatcccccccccccggcccaagcgggccgcctacctggccagccgggccgccgagcggcccgtgcccgccccccggaagcccacccccgccccccggaccgccttccggaacaagctgcccctgaccttcggcgacttcgacgagcacgaggtggacgccctggccagcggcatcaccttcggcgacttcgacgacgtgctgcggctgggccgggccggcgccatgaactacatccccacccagaccttctacggccggcggtggcggccccggcccgccgcccggccctggcccctgcaggccacccccgtggcccccgtggtgcccgacttccaggcccagcagatgcagcagctgatcagcgccgtgaacgccctgaccatgcggcagaacgccatcgcccccgcccggccccccaagcccaagaagaagaagaccaccaagcccaagcccaagacccagcccaagaagatcaacggcaagacccagcagcagaagaagaaggacaagcaggccgacaagaagaagaagaagcccggcaagcgggagcggatgtgcatgaagatcgagaacgactgcatcttcgaggtgaagcacgagggcaaggtgaccggctacgcctgcctggtgggcgacaaggtgatgaagcccgcccacgtgaagggcgtgatcgacaacgccgacctggccaagctggccttcaagaagagcagcaagtacgacctggagtgcgcccagatccccgtgcacatgcggagcgacgccagcaagtacacccacgagaagcccgagggccactacaactggcaccacggcgccgtgcagtacagcggcggccggttcaccatccccaccggcgccggcaagcccggcgacagcggccggcccatcttcgacaacaagggccgggtggtggccatcgtgctgggcggcgccaacgagggcagccggaccgccctgagcgtggtgacctggaacaaggacatggtgacccgggtgacccccgagggcagcgaggagtggagcgcccccctgatcaccgccatgtgcgtgctggccaacgccaccttcccctgcttccagcccccctgcgtgccctgctgctacgagaacaacgccgaggccaccctgcggatgctggaggacaacgtggaccggcccggctactacgacctgctgcaggccgccctgacctgccggaacggcacccggcaccggcggagcgtgagccagcacttcaacgtgtacaaggccacccggccctacatcgcctactgcgccgactgcggcgccggccacagctgccacagccccgtggccatcgaggccgtgcggagcgaggccaccgacggcatgctgaagatccagttcagcgcccagatcggcatcgacaagagcgacaaccacgactacaccaagatccggtacgccgacggccacgccatcgagaacgccgtgcggagcagcctgaaggtggccaccagcggcgactgcttcgtgcacggcaccatgggccacttcatcctggccaagtgcccccccggcgagttcctgcaggtgagcatccaggacacccggaacgccgtgcgggcctgccggatccagtaccaccacgacccccagcccgtgggccgggagaagttcaccatccggccccactacggcaaggagatcccctgcaccacctaccagcagaccaccgccaagaccgtggaggagatcgacatgcacatgccccccgacacccccgaccggaccctgctgagccagcagagcggcaacgtgaagatcaccgtgggcggcaagaaggtgaagtacaactgcacctgcggcaccggcaacgtgggcaccaccaacagcgacatgaccatcaacacctgcctgatcgagcagtgccacgtgagcgtgaccgaccacaagaagtggcagttcaacagccccttcgtgccccgggccgacgagcccgcccggaagggcaaggtgcacatccccttccccctggacaacatcacctgccgggtgcccatggcccgggagcccaccgtgatccacggcaagcgggaggtgaccctgcacctgcaccccgaccaccccaccctgttcagctaccggaccctgggcgaggacccccagtaccacgaggagtgggtgaccgccgccgtggagcggaccatccccgtgcccgtggacggcatggagtaccactggggcaacaacgaccccgtgcggctgtggagccagctgaccaccgagggcaagccccacggctggccccaccagatcgtgcagtactactacggcctgtaccccgccgccaccgtgagcgccgtggtgggcatgagcctgctggccctgatcagcatcttcgccagctgctacatgctggtggccgcccggagcaagtgcctgaccccctacgccctgacccccggcgccgccgtgccctggaccctgggcatcctgtgctgcgccccccgggcccacgccgccagcgtggccgagaccatggcctacctgtgggaccagaaccaggccctgttctggctggagttcgccgcccccgtggcctgcatcctgatcatcacctactgcctgcggaacgtgctgtgctgctgcaagagcctgagcttcctggtgctgctgagcctgggcgccaccgcccgggcctacgagcacagcaccgtgatgcccaacgtggtgggcttcccctacaaggcccacatcgagcggcccggctacagccccctgaccctgcagatgcaggtggtggagaccagcctggagcccaccctgaacctggagtacatcacctgcgagtacaagaccgtggtgcccagcccctacgtgaagtgctgcggcgccagcgagtgcagcaccaaggagaagcccgactaccagtgcaaggtgtacaccggcgtgtaccccttcatgtggggcggcgcctactgcttctgcgacagcgagaacacccagctgagcgaggcctacgtggaccggagcgacgtgtgccggcacgaccacgccagcgcctacaaggcccacaccgccagcctgaaggccaaggtgcgggtgatgtacggcaacgtgaaccagaccgtggacgtgtacgtgaacggcgaccacgccgtgaccatcggcggcacccagttcatcttcggccccctgagcagcgcctggacccccttcgacaacaagatcgtggtgtacaaggacgaggtgttcaaccaggacttccccccctacggcagcggccagcccggccggttcggcgacatccagagccggaccgtggagagcaacgacctgtacgccaacaccgccctgaagctggcccggcccagccccggcatggtgcacgtgccctacacccagacccccagcggcttcaagtactggctgaaggagaagggcaccgccctgaacaccaaggcccccttcggctgccagatcaagaccaaccccgtgcgggccatgaactgcgccgtgggcaacatccccgtgagcatgaacctgcccgacagcgccttcacccggatcgtggaggcccccaccatcatcgacctgacctgcaccgtggccacctgcacccacagcagcgacttcggcggcgtgctgaccctgacctacaagaccgacaagaacggcgactgcagcgtgcacagccacagcaacgtggccaccctgcaggaggccaccgccaaggtgaagaccgccggcaaggtgaccctgcacttcagcaccgccagcgccagccccagcttcgtggtgagcctgtgcagcgcccgggccacctgcagcgccagctgcgagccccccaaggaccacatcgtgccctacgccgccagccacagcaacgtggtgttccccgacatgagcggcaccgccctgagctgggtgcagaagatcagcggcggcctgggcgccttcgccatcggcgccatcctggtgctggtggtggtgacctgcatcggcctgcggcggtga

SEQ ID NO: 3 - DNA, 1611 bp, Synthetic construct
  source 1..1611: mol_type = other DNA
atggccgccaaggtgcacgtggacatcgaggccgacagccccttcatcaagagcctgcagaaggccttccccagcttcgaggtggagagcctgcaggtgacccccaacgaccacgccaacgcccgggccttcagccacctggccaccaagctgatcgagcaggagaccgacaaggacaccctgatcctggacatcggcagcgcccccagccggcggatgatgagcacccacaagtaccactgcgtgtgccccatgcggagcgccgaggaccccgagcggctggtgtgctacgccaagaagctggccgccgccagcggcaaggtgctggaccgggagatcgccggcaagatcaccgacctgcagaccgtgatggccacccccgacgccgagagccccaccttctgcctgcacaccgacgtgacctgccggaccgccgccgaggtggccgtgtaccaggacgtgtacgccgtgcacgcccccaccagcctgtaccaccaggccatgaagggcgtgcggaccgcctactggatcggcttcgacaccacccccttcatgttcgacgccctggccggcgcctaccccacctacgccaccaactgggccgacgagcaggtgctgcaggcccggaacatcggcctgtgcgccgccagcctgaccgagggccggctgggcaagctgagcatcctgcggaagaagcagctgaagccctgcgacaccgtgatgttcagcgtgggcagcaccctgtacaccgagagccggaagctgctgcggagctggcacctgcccagcgtgttccacctgaagggcaagcagagcttcacctgccggtgcgacaccatcgtgagctgcgagggctacgtggtgaagaagatcaccatgtgccccggcctgtacggcaagaccgtgggctacgccgtgacctaccacgccgagggcttcctggtgtgcaagaccaccgacaccgtgaagggcgagcgggtgagcttccccgtgtgcacctacgtgcccagcaccatctgcgaccagatgaccggcatcctggccaccgacgtgacccccgaggacgcccagaagctgctggtgggcctgaaccagcggatcgtggtgaacggccggacccagcggaacaccaacaccatgaagaactacctgctgcccatcgtggccgtggccttcagcaagtgggcccgggagtacaaggccgacctggacgacgagaagcccctgggcgtgcgggagcggagcctgacctgctgctgcctgtgggccttcaagacccggaagatgcacaccatgtacaagaagcccgacacccagaccatcgtgaaggtgcccagcgagttcaacagcttcgtgatccccagcctgtggagcaccggcctggccatccccgtgcggagccggatcaagatgctgctggccaagaagaccaagcgggagctgatccccgtgctggacgccagcagcgcccgggacgccgagcaggaggagaaggagcggctggaggccgagctgacccgggaggccctgccccccctggtgcccatcgcccccgccgagaccggcgtggtggacgtggacgtggaggagctggagtaccacgccggcgcc

SEQ ID NO: 4 - DNA, 2397 bp, Synthetic construct
  source 1..2397: mol_type = other DNA
ggcgtggtggagaccccccggagcgccctgaaggtgaccgcccagcccaacgacgtgctgctgggcaactacgtggtgctgagcccccagaccgtgctgaagagcagcaagctggcccccgtgcaccccctggccgagcaggtgaagatcatcacccacaacggccgggccggccggtaccaggtggacggctacgacggccgggtgctgctgccctgcggcagcgccatccccgtgcccgagttccaggccctgagcgagagcgccaccatggtgtacaacgagcgggagttcgtgaaccggaagctgtaccacatcgccgtgcacggccccagcctgaacaccgacgaggagaactacgagaaggtgcgggccgagcggaccgacgccgagtacgtgttcgacgtggacaagaagtgctgcgtgaagcgggaggaggccagcggcctggtgctggtgggcgagctgaccaacccccccttccacgagttcgcctacgagggcctgaagatccggcccagcgccccctacaagaccaccgtggtgggcgtgttcggcgtgcccggcagcggcaagagcgccatcatcaagagcctggtgaccaagcacgacctggtgaccagcggcaagaaggagaactgccaggagatcgtgaacgacgtgaagaagcaccggggcctggacatccaggccaagaccgtggacagcatcctgctgaacggctgccggcgggccgtggacatcctgtacgtggacgaggccttcgcctgccaccccggcaccctgctggccctgatcgccctggtgaagccccggagcaaggtggtgctgtgcggcgaccccaagcagtgcggcttcttcaacatgatgcagctgaaggtgaacttcaaccacaacatctgcaccgaggtgtgccacaagagcatcagccggcggtgcacccggcccgtgaccgccatcgtgagcaccctgcactacggcggcaagatgcggaccaccaacccctgcaacaagcccatcatcatcgacaccaccggccagaccaagcccaagcccggcgacatcgtgctgacctgcttccggggctgggtgaagcagctgcagctggactaccggggccacgaggtgatgaccgccgccgccagccagggcctgacccggaagggcgtgtacgccgtgcggcagaaggtgaacgagaaccccctgtacgcccccgccagcgagcacgtgaacgtgctgctgacccggaccgaggaccggctggtgtggaagaccctggccggcgacccctggatcaaggtgctgagcaacatcccccagggcaacttcaccgccaccctggaggagtggcaggaggagcacgacaagatcatgaaggtgatcgagggccccgccgcccccgtggacgccttccagaacaaggccaacgtgtgctgggccaagagcctggtgcccgtgctggacaccgccggcatccggctgaccgccgaggagtggagcaccatcatcaccgccttcaaggaggaccgggcctacagccccgtggtggccctgaacgagatctgcaccaagtactacggcgtggacctggacagcggcctgttcagcgcccccaaggtgagcctgtactacgagaacaaccactgggacaaccggcccggcggccggatgtacggcttcaacgccgccaccgccgcccggctggaggcccggcacaccttcctgaagggccagtggcacaccggcaagcaggccgtgatcgccgagcggaagatccagcccctgagcgtgctggacaacgtgatccccatcaaccggcggctgccccacgccctggtggccgagtacaagaccgtgaagggcagccgggtggagtggctggtgaacaaggtgcggggctaccacgtgctgctggtgagcgagtacaacctggccctgccccggcgggacgtgacctggctgagccccctgaacgtgaccggcgccgaccggtgctacgacctgagcctgggcctgcccgccgacgccggccggttcgacctggtgttcgtgaacatccacaccgagttccggatccaccactaccagcagtgcgtggaccacgccatgaagctgcagatgctgggcggcgacgccctgcggctgctgaagcccggcggcagcctgctgatgcgggcctacggctacgccgacaagatcagcgaggccgtggtgagcagcctgagccggaagttcagcagcgcccgggtgctgcggcccgactgcgtgaccagcaacaccgaggtgttcctgctgttcagcaacttcgacaacggcaagcggcccagcaccctgcaccagatgaacaccaagctgagcgccgtgtacgccggcgaggccatgcacaccgccggctgc

SEQ ID NO: 5 - DNA, 1446 bp, Synthetic construct
  source 1..1446: mol_type = other DNA
gcccccagctaccgggtgaagcgggccgacatcgccacctgcaccgaggccgccgtggtgaacgccgccaacgcccggggcaccgtgggcgacggcgtgtgccgggccgtggccaagaagtggcccagcgccttcaagggcgaggccacccccgtgggcaccatcaagaccgtgatgtgcggcagctaccccgtgatccacgccgtggcccccaacttcagcgccaccaccgaggccgagggcgaccgggagctggccgccgtgtaccgggccgtggccgccgaggtgaaccggctgagcctgagcagcgtggccatccccctgctgagcaccggcgtgttcagcggcggccgggaccggctgcagcagagcctgaaccacctgttcaccgccatggacgccaccgacgccgacgtgaccatctactgccgggacaagagctgggagaagaagatccaggaggccatcgacatgcggaccgccgtggagctgctgaacgacgacgtggagctgaccaccgacctggtgcgggtgcaccccgacagcagcctggtgggccggaagggctacagcaccaccgacggcagcctgtacagctacttcgagggcaccaagttcaaccaggccgccatcgacatggccgagatcctgaccctgtggccccggctgcaggaggccaacgagcagatctgcctgtacgccctgggcgagaccatggacaacatccggagcaagtgccccgtgaacgacagcgacagcagcacccccccccggaccgtgccctgcctgtgccggtacgccatgaccgccgagcggatcgcccggctgcggagccaccaggtgaagagcatggtggtgtgcagcagcttccccctgcccaagtaccacgtggacggcgtgcagaaggtgaagtgcgagaaggtgctgctgttcgaccccaccgtgcccagcgtggtgagcccccggaagtacgccgccagcaccaccgaccacagcgaccggagcctgcggggcttcgacctggactggaccaccgacagcagcagcaccgccagcgacaccatgagcctgcccagcctgcagagctgcgacatcgacagcatctacgagcccatggcccccatcgtggtgaccgccgacgtgcaccccgagcccgccggcatcgccgacctggccgccgacgtgcaccccgagcccgccgaccacgtggacctggagaaccccatcccccccccccggcccaagcgggccgcctacctggccagccgggccgccgagcggcccgtgcccgccccccggaagcccacccccgccccccggaccgccttccggaacaagctgcccctgaccttcggcgacttcgacgagcacgaggtggacgccctggccagcggcatcaccttcggcgacttcgacgacgtgctgcggctgggccgggccggcgcc

SEQ ID NO: 6 - DNA, 3762 bp, Synthetic construct
  source 1..3762: mol_type = other DNA
atgaactacatccccacccagaccttctacggccggcggtggcggccccggcccgccgcccggccctggcccctgcaggccacccccgtggcccccgtggtgcccgacttccaggcccagcagatgcagcagctgatcagcgccgtgaacgccctgaccatgcggcagaacgccatcgcccccgcccggccccccaagcccaagaagaagaagaccaccaagcccaagcccaagacccagcccaagaagatcaacggcaagacccagcagcagaagaagaaggacaagcaggccgacaagaagaagaagaagcccggcaagcgggagcggatgtgcatgaagatcgagaacgactgcatcttcgaggtgaagcacgagggcaaggtgaccggctacgcctgcctggtgggcgacaaggtgatgaagcccgcccacgtgaagggcgtgatcgacaacgccgacctggccaagctggccttcaagaagagcagcaagtacgacctggagtgcgcccagatccccgtgcacatgcggagcgacgccagcaagtacacccacgagaagcccgagggccactacaactggcaccacggcgccgtgcagtacagcggcggccggttcaccatccccaccggcgccggcaagcccggcgacagcggccggcccatcttcgacaacaagggccgggtggtggccatcgtgctgggcggcgccaacgagggcagccggaccgccctgagcgtggtgacctggaacaaggacatggtgacccgggtgacccccgagggcagcgaggagtggagcgcccccctgatcaccgccatgtgcgtgctggccaacgccaccttcccctgcttccagcccccctgcgtgccctgctgctacgagaacaacgccgaggccaccctgcggatgctggaggacaacgtggaccggcccggctactacgacctgctgcaggccgccctgacctgccggaacggcacccggcaccggcggagcgtgagccagcacttcaacgtgtacaaggccacccggccctacatcgcctactgcgccgactgcggcgccggccacagctgccacagccccgtggccatcgaggccgtgcggagcgaggccaccgacggcatgctgaagatccagttcagcgcccagatcggcatcgacaagagcgacaaccacgactacaccaagatccggtacgccgacggccacgccatcgagaacgccgtgcggagcagcctgaaggtggccaccagcggcgactgcttcgtgcacggcaccatgggccacttcatcctggccaagtgcccccccggcgagttcctgcaggtgagcatccaggacacccggaacgccgtgcgggcctgccggatccagtaccaccacgacccccagcccgtgggccgggagaagttcaccatccggccccactacggcaaggagatcccctgcaccacctaccagcagaccaccgccaagaccgtggaggagatcgacatgcacatgccccccgacacccccgaccggaccctgctgagccagcagagcggcaacgtgaagatcaccgtgggcggcaagaaggtgaagtacaactgcacctgcggcaccggcaacgtgggcaccaccaacagcgacatgaccatcaacacctgcctgatcgagcagtgccacgtgagcgtgaccgaccacaagaagtggcagttcaacagccccttcgtgccccgggccgacgagcccgcccggaagggcaaggtgcacatccccttccccctggacaacatcacctgccgggtgcccatggcccgggagcccaccgtgatccacggcaagcgggaggtgaccctgcacctgcaccccgaccaccccaccctgttcagctaccggaccctgggcgaggacccccagtaccacgaggagtgggtgaccgccgccgtggagcggaccatccccgtgcccgtggacggcatggagtaccactggggcaacaacgaccccgtgcggctgtggagccagctgaccaccgagggcaagccccacggctggccccaccagatcgtgcagtactactacggcctgtaccccgccgccaccgtgagcgccgtggtgggcatgagcctgctggccctgatcagcatcttcgccagctgctacatgctggtggccgcccggagcaagtgcctgaccccctacgccctgacccccggcgccgccgtgccctggaccctgggcatcctgtgctgcgccccccgggcccacgccgccagcgtggccgagaccatggcctacctgtgggaccagaaccaggccctgttctggctggagttcgccgcccccgtggcctgcatcctgatcatcacctactgcctgcggaacgtgctgtgctgctgcaagagcctgagcttcctggtgctgctgagcctgggcgccaccgcccgggcctacgagcacagcaccgtgatgcccaacgtggtgggcttcccctacaaggcccacatcgagcggcccggctacagccccctgaccctgcagatgcaggtggtggagaccagcctggagcccaccctgaacctggagtacatcacctgcgagtacaagaccgtggtgcccagcccctacgtgaagtgctgcggcgccagcgagtgcagcaccaaggagaagcccgactaccagtgcaaggtgtacaccggcgtgtaccccttcatgtggggcggcgcctactgcttctgcgacagcgagaacacccagctgagcgaggcctacgtggaccggagcgacgtgtgccggcacgaccacgccagcgcctacaaggcccacaccgccagcctgaaggccaaggtgcgggtgatgtacggcaacgtgaaccagaccgtggacgtgtacgtgaacggcgaccacgccgtgaccatcggcggcacccagttcatcttcggccccctgagcagcgcctggacccccttcgacaacaagatcgtggtgtacaaggacgaggtgttcaaccaggacttccccccctacggcagcggccagcccggccggttcggcgacatccagagccggaccgtggagagcaacgacctgtacgccaacaccgccctgaagctggcccggcccagccccggcatggtgcacgtgccctacacccagacccccagcggcttcaagtactggctgaaggagaagggcaccgccctgaacaccaaggcccccttcggctgccagatcaagaccaaccccgtgcgggccatgaactgcgccgtgggcaacatccccgtgagcatgaacctgcccgacagcgccttcacccggatcgtggaggcccccaccatcatcgacctgacctgcaccgtggccacctgcacccacagcagcgacttcggcggcgtgctgaccctgacctacaagaccgacaagaacggcgactgcagcgtgcacagccacagcaacgtggccaccctgcaggaggccaccgccaaggtgaagaccgccggcaaggtgaccctgcacttcagcaccgccagcgccagccccagcttcgtggtgagcctgtgcagcgcccgggccacctgcagcgccagctgcgagccccccaaggaccacatcgtgccctacgccgccagccacagcaacgtggtgttccccgacatgagcggcaccgccctgagctgggtgcagaagatcagcggcggcctgggcgccttcgccatcggcgccatcctggtgctggtggtggtgacctgcatcggcctgcggcggtga

SEQ ID NO: 7 - DNA, 221 bp, Alphavirus
  source 1..221: mol_type = genomic DNA
atggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgat

SEQ ID NO: 8 - DNA, 984 bp, Alphavirus
  source 1..984: mol_type = genomic DNA
ggacgtatgcaggcatgatcacgcatctgcttacaaagcccatacagcatcgctgaaggccaaagtgagggttatgtacggcaacgtaaaccagactgtggatgtttacgtgaacggagaccatgccgtcacgatagggggtactcagttcatattcgggccgctgtcatcggcctggaccccgttcgacaacaagatagtcgtgtacaaagacgaagtgttcaatcaggacttcccgccgtacggatctgggcaaccagggcgcttcggcgacatccaaagcagaacagtggagagtaacgacctgtacgcgaacacggcactgaagctggcacgcccttcacccggcatggtccatgtaccgtacacacagacaccttcagggttcaaatattggctaaaggaaaaagggacagccctaaatacgaaggctccttttggctgccaaatcaaaacgaaccctgtcagggccatgaactgcgccgtgggaaacatccctgtctccatgaatttgcctgacagcgcctttacccgcattgtcgaggcgccgaccatcattgacctgacttgcacagtggctacctgtacgcactcctcggatttcggcggcgtcttgacactgacgtacaagaccgacaagaacggggactgctctgtacactcgcactctaacgtagctactctacaggaggccacagcaaaagtgaagacagcaggtaaggtgaccttacacttctccacggcaagcgcatcaccttcttttgtggtgtcgctatgcagtgctagggccacctgttcagcgtcgtgtgagcccccgaaagaccacatagtcccatatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagata

SEQ ID NO: 9 - DNA, 64 bp, synthetic construct
  source 1..64: mol_type = other DNA
taatacgactcactatagggaaataagagagaaaagaagagtaagaagaaatataagagccacc

SEQ ID NO: 10 - DNA, 389 bp, synthetic construct
  source 1..389: mol_type = other DNA
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctggagctagcgctgccttctgcggggcttgccttctggccatgcccttcttctctcccttgcacctgtacctcttggtctttgaataaagcctgagtaggaagt

SEQ ID NO: 11 - DNA, 9216 bp, Alphavirus
  source 1..9216: mol_type = other DNA
atggccgccaaagtgcatgttgatattgaggctgacagcccattcatcaagtctttgcagaaggcatttccgtcgttcgaggtggagtcattgcaggtcacaccaaatgaccatgcaaatgccagagcattttcgcacctggctaccaaattgatcgagcaggagactgacaaagacacactcatcttggatatcggcagtgcgccttccaggagaatgatgtctacgcacaaataccactgcgtatgccctatgcgcagcgcagaagaccccgaaaggctcgtatgctacgcaaagaaactggcagcggcctccgggaaggtgctggatagagagatcgcaggaaaaatcaccgacctgcagaccgtcatggctacgccagacgctgaatctcctaccttttgcctgcatacagacgtcacgtgtcgtacggcagccgaagtggccgtataccaggacgtgtatgctgtacatgcaccaacatcgctgtaccatcaggcgatgaaaggtgtcagaacggcgtattggattgggtttgacaccaccccgtttatgtttgacgcgctagcaggcgcgtatccaacctacgccacaaactgggccgacgagcaggtgttacaggccaggaacataggactgtgtgcagcatccttgactgagggaagactcggcaaactgtccattctccgcaagaagcaattgaaaccttgcgacacagtcatgttctcggtaggatctacattgtacactgagagcagaaagctactgaggagctggcacttaccctccgtattccacctgaaaggtaaacaatcctttacctgtaggtgcgataccatcgtatcatgtgaagggtacgtagttaagaaaatcactatgtgccccggcctgtacggtaaaacggtagggtacgccgtgacgtatcacgcggagggattcctagtgtgcaagaccacagacactgtcaaaggagaaagagtctcattccctgtatgcacctacgtcccctcaaccatctgtgatcaaatgactggcatactagcgaccgacgtcacaccggaggacgcacagaagttgttagtgggattgaatcagaggatagttgtgaacggaagaacacagcgaaacactaacacgatgaagaactatctgcttccgattgtggccgtcgcatttagcaagtgggcgagggaatacaaggcagaccttgatgatgaaaaacctctgggtgtccgagagaggtcacttacttgctgctgcttgtgggcatttaaaacgaggaagatgcacaccatgtacaagaaaccagacacccagacaatagtgaaggtgccttcagagtttaactcgttcgtcatcccgagcctatggtctacaggcctcgcaatcccagtcagatcacgcattaagatgcttttggccaagaagaccaagcgagagttaatacctgttctcgacgcgtcgtcagccagggatgctgaacaagaggagaaggagaggttggaggccgagctgactagagaagccttaccacccctcgttcccatcgcgccggcggagacgggagtcgtcgacgtcgacgttgaagaactagagtatcacgcaggtgcaggggtcgtggaaacacctcgcagcgcgttgaaagtcaccgcacagccgaacgacgtactactaggaaattacgtagttctgtccccgcagaccgtgctcaagagctccaagttggcccccgtgcaccctctagcagagcaggtgaaaataataacacataacgggagggccggccgttaccaggtcgacggatatgacggcagggtcctactaccatgtggatcggccattccggtccctgagtttcaagctttgagcgagagcgccactatggtgtacaacgaaagggagttcgtcaacaggaaactataccatattgccgttcacggaccgtcgctgaacaccgacgaggagaactacgagaaagtcagagctgaaagaactgacgccgagtacgtgttcgacgtagataaaaaatgctgcgtcaagagagaggaagcgtcgggtttggtgttggtgggagagctaaccaaccccccgttccatgaattcgcctacgaagggctgaagatcaggccgtcggcaccatataagactacagtagtaggagtctttggggttccgggatcaggcaagtctgctattattaagagcctcgtgaccaaacacgatctggtcaccagcggcaagaaggagaactgccaggaaatagtcaacgacgtgaagaagcaccgcggactggacatccaggcaaaaacagtggactccatcctgctaaacgggtgtcgtcgtgccgtggacatcctatatgtggacgaggctttcgcttgccattccggtactctgctagccctaattgctcttgttaaacctcggagcaaagtggtgttatgcggagaccccaagcaatgcggattcttcaatatgatgcagcttaaggtgaacttcaaccacaacatctgcactgaagtatgtcataaaagtatatccagacgttgcacgcgtccagtcacggccatcgtgtctacgttgcactacggaggcaagatgcgcacgaccaacccgtgcaacaaacccataatcatagacaccacaggacagaccaagcccaagccaggagacatcgtgttaacatgcttccgaggctgggtaaagcagctgcagttggactaccgtggacacgaagtcatgacagcagcagcatctcagggcctcacccgcaaaggggtatacgccgtaaggcagaaggtgaatgaaaatcccttgtatgcccctgcgtcggagcacgtgaatgtactgctgacgcgcactgaggataggctggtgtggaaaacgctggccggcgatccctggattaaggtcctatcaaacattccacagggtaactttacggccacattggaagaatggcaagaagaacacgacaaaataatgaaggtgattgaaggaccggctgcgcctgtggacgcgttccagaacaaagcgaacgtgtgttgggcgaaaagcctggtgcctgtcctggacactgccggaatcagattgacagcagaggagtggagcaccataattacagcatttaaggaggacagagcttactctccagtggtggccttgaatgaaatttgcaccaagtactatggagttgacctggacagtggcctgttttctgccccgaaggtgtccctgtattacgagaacaaccactgggataacagacctggtggaaggatgtatggattcaatgccgcaacagctgccaggctggaagctagacataccttcctgaaggggcagtggcatacgggcaagcaggcagttatcgcagaaagaaaaatccaaccgctttctgtgctggacaatgtaattcctatcaaccgcaggctgccgcacgccctggtggctgagtacaagacggttaaaggcagtagggttgagtggctggtcaataaagtaagagggtaccacgtcctgctggtgagtgagtacaacctggctttgcctcgacgcagggtcacttggttgtcaccgctgaatgtcacaggcgccgataggtgctacgacctaagtttaggactgccggctgacgccggcaggttcgacttggtctttgtgaacattcacacggaattcagaatccaccactaccagcagtgtgtcgaccacgccatgaagctgcagatgcttgggggagatgcgctacgactgctaaaacccggcggcagcctcttgatgagagcttacggatacgccgataaaatcagcgaagccgttgtttcctccttaagcagaaagttctcgtctgcaagagtgttgcgcccggattgtgtcaccagcaatacagaagtgttcttgctgttctccaactttgacaacggaaagagaccctctacgctacaccagatgaataccaagctgagtgccgtgtatgccggagaagccatgcacacggccgggtgtgcaccatcctacagagttaagagagcagacatagccacgtgcacagaagcggctgtggttaacgcagctaacgcccgtggaactgtaggggatggcgtatgcagggccgtggcgaagaaatggccgtcagcctttaagggagaagcaacaccagtgggcacaattaaaacagtcatgtgcggctcgtaccccgtcatccacgctgtagcgcctaatttctctgccacgactgaagcggaaggggaccgcgaattggccgctgtctaccgggcagtggccgccgaagtaaacagactgtcactgagcagcgtagccatcccgctgctgtccacaggagtgttcagcggcggaagagataggctgcagcaatccctcaaccatctattcacagcaatggacgccacggacgctgacgtgaccatctactgcagagacaaaagttgggagaagaaaatccaggaagccatagacatgaggacggctgtggagttgctcaatgatgacgtggagctgaccacagacttggtgagagtgcacccggacagcagcctggtgggtcgtaagggctacagtaccactgacgggtcgctgtactcgtactttgaaggtacgaaattcaaccaggctgctattgatatggcagagatactgacgttgtggcccagactgcaagaggcaaacgaacagatatgcctatacgcgctgggcgaaacaatggacaacatcagatccaaatgtccggtgaacgattccgattcatcaacacctcccaggacagtgccctgcctgtgccgctacgcaatgacagcagaacggatcgcccgccttaggtcacaccaagttaaaagcatggtggtttgctcatcttttcccctcccgaaataccatgtagatggggtgcagaaggtaaagtgcgagaaggttctcctgttcgacccgacggtaccttcagtggttagtccgcggaagtatgccgcatctacgacggaccactcagatcggtcgttacgagggtttgacttggactggaccaccgactcgtcttccactgccagcgataccatgtcgctacccagtttgcagtcgtgtgacatcgactcgatctacgagccaatggctcccatagtagtgacggctgacgtacaccctgaacccgcaggcatcgcggacctggcggcagatgtgcatcctgaacccgcagaccatgtggacctcgagaacccgattcctccaccgcgcccgaagagagctgcataccttgcctcccgcgcggcggagcgaccggtgccggcgccgagaaagccgacgcctgccccaaggactgcgtttaggaacaagctgcctttgacgttcggcgactttgacgagcacgaggtcgatgcgttggcctccgggattactttcggagacttcgacgacgtcctgcgactaggccgcgcgggtgcaatgaattacatccctacgcaaacgttttacggccgccggtggcgcccgcgcccggcggcccgtccttggccgttgcaggccactccggtggctcccgtcgtccccgacttccaggcccagcagatgcagcaactcatcagcgccgtaaatgcgctgacaatgagacagaacgcaattgctcctgctaggcctcccaaaccaaagaagaagaagacaaccaaaccaaagccgaaaacgcagcccaagaagatcaacggaaaaacgcagcagcaaaagaagaaagacaagcaagccgacaagaagaagaagaaacccggaaaaagagaaagaatgtgcatgaagattgaaaatgactgtatcttcgaagtcaaacacgaaggaaaggtcactgggtacgcctgcctggtgggcgacaaagtcatgaaacctgcccacgtgaaaggagtcatcgacaacgcggacctggcaaagctagctttcaagaaatcgagcaagtatgaccttgagtgtgcccagataccagttcacatgaggtcggatgcctcaaagtacacgcatgagaagcccgagggacactataactggcaccacggggctgttcagtacagcggaggtaggttcactataccgacaggagcgggcaaaccgggagacagtggccggcccatctttgacaacaaggggagggtagtcgctatcgtcctgggcggggccaacgagggctcacgcacagcactgtcggtggtcacctggaacaaagatatggtgactagagtgacccccgaggggtccgaagagtggtccgccccgctgattactgccatgtgtgtccttgccaatgctaccttcccgtgcttccagcccccgtgtgtaccttgctgctatgaaaacaacgcagaggccacactacggatgctcgaggataacgtggataggccagggtactacgacctccttcaggcagccttgacgtgccgaaacggaacaagacaccggcgcagcgtgtcgcaacacttcaacgtgtataaggctacacgcccttacatcgcgtactgcgccgactgcggagcagggcactcgtgtcatagccccgtagcaattgaagcggtcaggtccgaagctaccgacgggatgctgaagattcagttctcggcacaaattggcatagataagagtgacaatcatgactacacgaagataaggtacgcagacgggcacgccattgagaatgccgtccggtcatctttgaaggtagccacctccggagactgtttcgtccatggcacaatgggacatttcatactggcaaagtgcccaccgggtgaattcctgcaggtctcgatccaggacaccagaaacgcggtccgtgcctgcagaatacaatatcatcatgaccctcaaccggtgggtagagaaaaatttacaattagaccacactatggaaaagagatcccttgcaccacttatcaacagaccacagcgaagaccgtggaggaaatcgacatgcatatgccgccagatacgccggacaggacgttgctatcacagcaatctggcaatgtaaagatcacagtcggaggaaagaaggtgaaatacaactgcacctgtggaaccggaaacgttggcactactaattcggacatgacgatcaacacgtgtctaatagagcagtgccacgtctcagtgacggaccataagaaatggcagttcaactcacctttcgtcccgagagccgacgaaccggctagaaaaggcaaagtccatatcccattcccgttggacaacatcacatgcagagttccaatggcgcgcgaaccaaccgtcatccacggcaaaagagaagtgacactgcaccttcacccagatcatcccacgctcttttcctaccgcacactgggtgaggacccgcagtatcacgaggaatgggtgacagcggcggtggaacggaccatacccgtaccagtggacgggatggagtaccactggggaaacaacgacccagtgaggctttggtctcaactcaccactgaagggaaaccgcacggctggccgcatcagatcgtacagtactactatgggctttacccggccgctacagtatccgcggtcgtcgggatgagcttactggcgttgatatcgatcttcgcgtcgtgctacatgctggttgcggcccgcagtaagtgcttgaccccttatgctttaacaccaggagctgcagttccgtggacgctggggatactctgctgcgccccgcgggcgcacgcagctagtgtggcagagactatggcctacttgtgggaccaaaaccaagcgttgttctggttggagtttgcggcccctgttgcctgcatcctcatcatcacgtattgcctcagaaacgtgctgtgttgctgtaagagcctttcttttttagtgctactgagcctcggggcaaccgccagagcttacgaacattcgacagtaatgccgaacgtggtggggttcccgtataaggctcacattgaaaggccaggatatagccccctcactttgcagatgcaggttgttgaaaccagcctcgaaccaacccttaatttggaatacataacctgtgagtacaagacggtcgtcccgtcgccgtacgtgaagtgctgcggcgcctcagagtgctccactaaagagaagcctgactaccaatgcaaggtttacacaggcgtgtacccgttcatgtggggaggggcatattgcttctgcgactcagaaaacacgcaactcagcgaggcgtacgtcgatcgatcggacgtatgcaggcatgatcacgcatctgcttacaaagcccatacagcatcgctgaaggccaaagtgagggttatgtacggcaacgtaaaccagactgtggatgtttacgtgaacggagaccatgccgtcacgatagggggtactcagttcatattcgggccgctgtcatcggcctggaccccgttcgacaacaagatagtcgtgtacaaagacgaagtgttcaatcaggacttcccgccgtacggatctgggcaaccagggcgcttcggcgacatccaaagcagaacagtggagagtaacgacctgtacgcgaacacggcactgaagctggcacgcccttcacccggcatggtccatgtaccgtacacacagacaccttcagggttcaaatattggctaaaggaaaaagggacagccctaaatacgaaggctccttttggctgccaaatcaaaacgaaccctgtcagggccatgaactgcgccgtgggaaacatccctgtctccatgaatttgcctgacagcgcctttacccgcattgtcgaggcgccgaccatcattgacctgacttgcacagtggctacctgtacgcactcctcggatttcggcggcgtcttgacactgacgtacaagaccgacaagaacggggactgctctgtacactcgcactctaacgtagctactctacaggaggccacagcaaaagtgaagacagcaggtaaggtgaccttacacttctccacggcaagcgcatcaccttcttttgtggtgtcgctatgcagtgctagggccacctgttcagcgtcgtgtgagcccccgaaagaccacatagtcccatatgcggctagccacagtaacgtagtgtttccagacatgtcgggcaccgcactatcatgggtgcagaaaatctcgggtggtctgggggccttcgcaatcggcgctatcctggtgctggttgtggtcacttgcattgggctccgcagataa

SEQ ID NO: 12 - DNA, 27 bp, synthetic construct
  source 1..27: mol_type = other DNA
ttggaccctcgtacagaagctaatacg

SEQ ID NO: 13 - DNA, 149 bp, synthetic construct
  source 1..149: mol_type = other DNA
ttttttttttttttttttttttttttttttttttttttttttttttttttttttttttttttttttttttttttttttttttttttttttttttttttttttttttttttttttttttttcttcctactcaggctttattcaaagacca

SEQ ID NO: 14 - DNA, 879 bp, synthetic construct
  source 1..879: mol_type = other DNA
atgcccctgcacgtgaagtggccgttccccgcggtgccgccgctcacctggaccctggccagcagcgtcgtcatgggcctcgtgggcacctacagctgcttctggaccaagtacatgaaccacctgaccgtgcacaaccgcgaggtgctgtacgagctcatcgagaagcgcggcccggccacgcccctcatcaccgtgtccaaccaccagtcctgcatggacgacccccacctctgggggatcctgaagctccgccacatctggaacctgaagctcatgcgctggacccccgccgccgccgacatctgcttcaccaaggagctccactcccacttcttcagcctcggcaagtgcgtgcccgtgtgccgcggcgccgagttcttccaggccgagaacgaggggaagggcgtcctcgacaccggccgccacatgcccggcgccggcaagcgccgcgagaagggcgacggcgtctaccagaaggggatggacttcatcctcgagaagctcaaccacggggactgggtgcacatcttccccgaggggaaggtgaacatgtcctccgagttcctgcgcttcaagtggggcatcgggcgcctgatcgccgagtgccacctcaaccccatcatcctgcccctgtggcacgtcggcatgaacgacgtcctccccaactccccgccctacttcccccgcttcggccagaagatcaccgtgctgatcgggaagcccttctccgccctgcccgtcctcgagcggctccgggcggagaacaagtcggccgtggagatgcggaaggccctgacggacttcatccaggaggagttccagcacctgaagacccaggccgagcagctccacaaccacctccagcccgggcgctag

SEQ ID NO: 15 - DNA, 948 bp, Synthetic construct
  source 1..948: mol_type = other DNA
atggtgagcaagggcgaggagctgttcaccggcgtggtgcccatcctggtggagctggacggcgacgtgaacggccacaagttcagcgtgagcggcgagggcgagggcgacgccacctacggcaagctgaccctgaagttcatctgcaccaccggcaagctgcccgtgccctggcccaccctggtgaccaccctgacctacggcgtgcagtgcttcagccggtaccccgaccacatgaagcagcacgacttcttcaagagcgccatgcccgagggctacgtgcaggagcggaccatcttcttcaaggacgacggcaactacaagacccgggccgaggtgaagttcgagggcgacaccctggtgaaccggatcgagctgaagggcatcgacttcaaggaggacggcaacatcctgggccacaagctggagtacaactacaacagccacaacgtgtacatcatggccgacaagcagaagaacggcatcaaggtgaacttcaagatccggcacaacatcgaggacggcagcgtgcagctggccgaccactaccagcagaacacccccatcggcgacggccccgtgctgctgcccgacaaccactacctgagcacccagagcgccctgagcaaggaccccaacgagaagcgggaccacatggtgctgctggagttcgtgaccgccgccggcatcaccctgggcatggacgagctgtacaagaagctgagccacggcttcccccccgaggtggaggagcaggacgacggcaccctgcccatgagctgcgcccaggagagcggcatggaccggcaccccgccgcctgcgccagcgcccggatcaacgtgcggagccgggccgaccccaagaagaagcggaaggtggaccccaagaagaagcggaaggtggaccccaagaagaagcggaaggtgggcagcaccggcagccggtga

SEQ ID NO: 16 - DNA, 4110 bp, Synthetic construct
  source 1..4110: mol_type = other DNA
atggacaagaagtacagcatcggcctggacatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccggaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgagccggaagctgatcaacggcatccgggacaagcagagcggcaagaccatcctggacttcctgaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctgaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaagaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagctga

SEQ ID NO: 17 - DNA, 456 bp, Synthetic construct
  source 1..456: mol_type = other DNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgagtttcttggctttatatatcttgtggaaaggacgaaacaccgaccactgaaggcctgaaaggttttagagctagaaatagcaagttaaaataaggctagtccgttatcaacttgaaaaagtggcaccgagtcggtgctttttttctagacccagctttcttgtacaaagttggcatta

SEQ ID NO: 18 - DNA, 3822 bp, Synthetic construct
  source 1..3822: mol_type = other DNA
atgttcgtgttcctggtgctgctgcccctggtgagcagccagtgcgtgaacctgaccacccggacccagctgccccccgcctacaccaacagcttcacccggggcgtgtactaccccgacaaggtgttccggagcagcgtgctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctgggcgtgtactaccacaagaacaacaagagctggatggagagcgagttccgggtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacacccccatcaacctggtgcgggacctgccccagggcttcagcgccctggagcccctggtggacctgcccatcggcatcaacatcacccggttccagaccctgctggccctgcaccggagctacctgacccccggcgacagcagcagcggctggaccgccggcgccgccgcctactacgtgggctacctgcagccccggaccttcctgctgaagtacaacgagaacggcaccatcaccgacgccgtggactgcgccctggaccccctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccggggcgacgaggtgcggcagatcgcccccggccagaccggcaagatcgccgactacaactacaagctgcccgacgacttcaccggctgcgtgatcgcctggaacagcaacaacctggacagcaaggtgggcggcaactacaactacctgtaccggctgttccggaagagcaacctgaagcccttcgagcgggacatcagcaccgagatctaccaggccggcagcaccccctgcaacggcgtggagggcttcaactgctacttccccctgcagagctacggcttccagcccaccaacggcgtgggctaccagccctaccgggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggccgggacatcgccgacaccaccgacgccgtgcgggacccccagaccctggagatcctggacatcaccccctgcagcttcggcggcgtgagcgtgatcacccccggcaccaacaccagcaaccaggtggccgtgctgtaccaggacgtgaactgcaccgaggtgcccgtggccatccacgccgaccagctgacccccacctggcgggtgtacagcaccggcagcaacgtgttccagacccgggccggctgcctgatcggcgccgagcacgtgaacaacagctacgagtgcgacatccccatcggcgccggcatctgcgccagctaccagacccagaccaacagcccccggcgggcccggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcaggacgtggtgaaccagaacgcccaggccctgaacaccctggtgaagcagctgagcagcaacttcggcgccatcagcagcgtgctgaacgacatcctgagccggctggacccccccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttcccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgaccggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacctga

SEQ ID NO: 19 - DNA, 459 bp, Synthetic construct
  source 1..459: mol_type = other DNA
atgtaccggatgcagctgctgagctgcatcgccctgagcctggccctggtgaccaacagcgcccccaccagcagcagcaccaagaagacccagctgcagctggagcacctgctgctggacctgcagatgatcctgaacggcatcaacaactacaagaaccccaagctgacccggatgctgaccttcaagttctacatgcccaagaaggccaccgagctgaagcacctgcagtgcctggaggaggagctgaagcccctggaggaggtgctgaacctggcccagagcaagaacttccacctgcggccccgggacctgatcagcaacatcaacgtgatcgtgctggagctgaagggcagcgagaccaccttcatgtgcgagtacgccgacgagaccgccaccatcgtggagttcctgaaccggtggatcaccttctgccagagcatcatcagcaccctgacc

SEQ ID NO: 20 - DNA, 783 bp, Synthetic construct
  source 1..783: mol_type = other DNA
atgtggctgttcatcgccagcgcccccttcgaggtggccgagggcgagaacgtgcacctgagcgtggtgtacctgcccgagaacctgtacagctacggctggtacaagggcaagaccgtggagcccaaccagctgatcgccgcctacgtgatcgacacccacgtgcggacccccggccccgcctacagcggccgggagaccatcagccccagcggcgacctgcacttccagaacgtgaccctggaggacaccggctactacaacctgcaggtgacctaccggaacagccagatcgagcaggccagccaccacctgcgggtgtacgagagcgtggcccagcccagcatccaggccagcagcaccaccgtgaccgagaagggcagcgtggtgctgacctgccacaccaacaacaccggcaccagcttccagtggatcttcaacaaccagcggctgcaggtgaccaagcggatgaagctgagctggttcaaccacgtgctgaccatcgaccccatccggcaggaggacgccggcgagtaccagtgcgaggtgagcaaccccgtgagcagcaaccggagcgaccccctgaagctgaccgtgaagagcgacgacaacaccctgggcatcctgatcggcgtgctggtgggcagcctgctggtggccgccctggtgtgcttcctgctgctgcggaagaccggccgggccagcgaccagagcgacttccgggagcagcagccccccgccagcacccccggccacggccccagcgacagcagcatcagctga

SEQ ID NO: 21 - DNA, 1500 bp, Synthetic construct
  source 1..1500: mol_type = other DNA
atgtggcggcccagcgacagcaccgtgtacgtgcccccccccaaccccgtgagcaaggtggtggccaccgacgcctacgtgacccggaccaacatcttctaccacgccagcagcagccggctgctggccgtgggccacccctacttcagcatcaagcgggccaacaagaccgtggtgcccaaggtgagcggctaccagtaccgggtgttcaaggtggtgctgcccgaccccaacaagttcgccctgcccgacagcagcctgttcgaccccaccacccagcggctggtgtgggcctgcaccggcctggaggtgggccggggccagcccctgggcgtgggcgtgagcggccaccccttcctgaacaagtacgacgacgtggagaacagcggcagcggcggcaaccccggccaggacaaccgggtgaacgtgggcatggactacaagcagacccagctgtgcatggtgggctgcgccccccccctgggcgagcactggggcaagggcaagcagtgcaccaacacccccgtgcaggccggcgactgcccccccctggagctgatcaccagcgtgatccaggacggcgacatggtggacaccggcttcggcgccatgaacttcgccgacctgcagaccaacaagagcgacgtgcccatcgacatctgcggcaccacctgcaagtaccccgactacctgcagatggccgccgacccctacggcgaccggctgttcttcttcctgcggaaggagcagatgttcgcccggcacttcttcaaccgggccggcgaggtgggcgagcccgtgcccgacaccctgatcatcaagggcagcggcaaccggaccagcgtgggcagcagcatctacgtgaacacccccagcggcagcctggtgagcagcgaggcccagctgttcaacaagccctactggctgcagaaggcccagggccacaacaacggcatctgctggggcaaccagctgttcgtgaccgtggtggacaccacccggagcaccaacatgaccctgtgcgccagcgtggccaccagcagcacctacaccaacagcgactacaaggagtacatgcggcacgtggaggagtacgacctgcagttcatcttccagctgtgcagcatcaccctgagcgccgaggtgatggcctacatccacaccatgaaccccagcgtgctggaggactggaacttcggcctgagccccccccccaacggcaccctggaggacacctaccggtacgtgcagagccaggccatcacctgccagaagcccacccccgagaaggagaagcccgacccctacaagaacctgagcttctgggaggtgaacctgaaggagaagttcagcagcgagctggaccagtaccccctgggccggaagttcctgctgcagagcggctaccggggccggagcagcatccggaccggcgtgaagcggcccgccgtgagcaaggccagcgccgcccccaagcggaagcgggccaagaccaagcgg

SEQ ID NO: 22 - DNA, 1506 bp, Synthetic construct
  source 1..1506: mol_type = other DNA
atgtggcggcccagcgacagcaccgtgtacgtgcccccccccaaccccgtgagcaaggtggtggccaccgacgcctacgtgaagcggaccaacatcttctaccacgccagcagcagccggctgctggccgtgggccacccctactacagcatcaagaaggtgaacaagaccgtggtgcccaaggtgagcggctaccagtaccgggtgttcaaggtggtgctgcccgaccccaacaagttcgccctgcccgacagcagcctgttcgaccccaccacccagcggctggtgtgggcctgcaccggcctggaggtgggccggggccagcccctgggcgtgggcgtgagcggccaccccctgctgaacaagtacgacgacgtggagaacagcggcggctacggcggcaaccccggccaggacaaccgggtgaacgtgggcatggactacaagcagacccagctgtgcatggtgggctgcgccccccccctgggcgagcactggggcaagggcacccagtgcagcaacaccagcgtgcagaacggcgactgcccccccctggagctgatcaccagcgtgatccaggacggcgacatggtggacaccggcttcggcgccatgaacttcgccgacctgcagaccaacaagagcgacgtgcccctggacatctgcggcaccgtgtgcaagtaccccgactacctgcagatggccgccgacccctacggcgaccggctgttcttctacctgcggaaggagcagatgttcgcccggcacttcttcaaccgggccggcaccgtgggcgagcccgtgcccgacgacctgctggtgaagggcggcaacaaccggagcagcgtggccagcagcatctacgtgcacacccccagcggcagcctggtgagcagcgaggcccagctgttcaacaagccctactggctgcagaaggcccagggccacaacaacggcatctgctggggcaaccacctgttcgtgaccgtggtggacaccacccggagcaccaacatgaccctgtgcgccagcgtgagcaagagcgccacctacaccaacagcgactacaaggagtacatgcggcacgtggaggagttcgacctgcagttcatcttccagctgtgcagcatcaccctgagcgccgaggtgatggcctacatccacaccatgaaccccagcgtgctggaggactggaacttcggcctgagccccccccccaacggcaccctggaggacacctaccggtacgtgcagagccaggccatcacctgccagaagcccacccccgagaaggagaagcaggacccctacaaggacatgagcttctgggaggtgaacctgaaggagaagttcagcagcgagctggaccagttccccctgggccggaagttcctgctgcagagcggctaccggggccggaccagcgcccggaccggcatcaagcggcccgccgtgagcaagcccagcaccgcccccaagcggaagcggaccaagaccaagaagtga

SEQ ID NO: 23 - DNA, 1518 bp, Synthetic construct
  source 1..1518: mol_type = other DNA
atgagcctgtggctgcccagcgaggccaccgtgtacctgccccccgtgcccgtgagcaaggtggtgagcaccgacgagtacgtggcccggaccaacatctactaccacgccggcaccagccggctgctggccgtgggccacccctacttccccatcaagaagcccaacaacaacaagatcctggtgcccaaggtgagcggcctgcagtaccgggtgttccggatccacctgcccgaccccaacaagttcggcttccccgacaccagcttctacaaccccgacacccagcggctggtgtgggcctgcgtgggcgtggaggtgggccggggccagcccctgggcgtgggcatcagcggccaccccctgctgaacaagctggacgacaccgagaacgccagcgcctacgccgccaacgccggcgtggacaaccgggagtgcatcagcatggactacaagcagacccagctgtgcctgatcggctgcaagccccccatcggcgagcactggggcaagggcagcccctgcaccaacgtggccgtgaaccccggcgactgcccccccctggagctgatcaacaccgtgatccaggacggcgacatggtggacaccggcttcggcgccatggacttcaccaccctgcaggccaacaagagcgaggtgcccctggacatctgcaccagcatctgcaagtaccccgactacatcaagatggtgagcgagccctacggcgacagcctgttcttctacctgcggcgggagcagatgttcgtgcggcacctgttcaaccgggccggcgccgtgggcgagaacgtgcccgacgacctgtacatcaagggcagcggcagcaccgccaacctggccagcagcaactacttccccacccccagcggcagcatggtgaccagcgacgcccagatcttcaacaagccctactggctgcagcgggcccagggccacaacaacggcatctgctggggcaaccagctgttcgtgaccgtggtggacaccacccggagcaccaacatgagcctgtgcgccgccatcagcaccagcgagaccacctacaagaacaccaacttcaaggagtacctgcggcacggcgaggagtacgacctgcagttcatcttccagctgtgcaagatcaccctgaccgccgacgtgatgacctacatccacagcatgaacagcaccatcctggaggactggaacttcggcctgcagcccccccccggcggcaccctggaggacacctaccggttcgtgaccagccaggccatcgcctgccagaagcacaccccccccgcccccaaggaggaccccctgaagaagtacaccttctgggaggtgaacctgaaggagaagttcagcgccgacctggaccagttccccctgggccggaagttcctgctgcaggccggcctgaaggccaagcccaagttcaccctgggcaagcggaaggccacccccaccaccagcagcaccagcaccaccgccaagcggaagaagcggaagctgtga

SEQ ID NO: 24 - DNA, 1707 bp, Synthetic construct
  source 1..1707: mol_type = other DNA
atgtgcctgtacacccgggtgctgatcctgcactaccacctgctgcccctgtacggccccctgtaccacccccggcccctgcccctgcacagcatcctggtgtacatggtgcacatcatcatctgcggccactacatcatcctgttcctgcggaacgtgaacgtgttccccatcttcctgcagatggccctgtggcggcccagcgacaacaccgtgtacctgcccccccccagcgtggcccgggtggtgaacaccgacgactacgtgacccccaccagcatcttctaccacgccggcagcagccggctgctgaccgtgggcaacccctacttccgggtgcccgccggcggcggcaacaagcaggacatccccaaggtgagcgcctaccagtaccgggtgttccgggtgcagctgcccgaccccaacaagttcggcctgcccgacaccagcatctacaaccccgagacccagcggctggtgtgggcctgcgccggcgtggagatcggccggggccagcccctgggcgtgggcctgagcggccaccccttctacaacaagctggacgacaccgagagcagccacgccgccaccagcaacgtgagcgaggacgtgcgggacaacgtgagcgtggactacaagcagacccagctgtgcatcctgggctgcgcccccgccatcggcgagcactgggccaagggcaccgcctgcaagagccggcccctgagccagggcgactgcccccccctggagctgaagaacaccgtgctggaggacggcgacatggtggacaccggctacggcgccatggacttcagcaccctgcaggacaccaagtgcgaggtgcccctggacatctgccagagcatctgcaagtaccccgactacctgcagatgagcgccgacccctacggcgacagcatgttcttctgcctgcggcgggagcagctgttcgcccggcacttctggaaccgggccggcaccatgggcgacaccgtgccccagagcctgtacatcaagggcaccggcatgcccgccagccccggcagctgcgtgtacagccccagccccagcggcagcatcgtgaccagcgacagccagctgttcaacaagccctactggctgcacaaggcccagggccacaacaacggcgtgtgctggcacaaccagctgttcgtgaccgtggtggacaccacccccagcaccaacctgaccatctgcgccagcacccagagccccgtgcccggccagtacgacgccaccaagttcaagcagtacagccggcacgtggaggagtacgacctgcagttcatcttccagctgtgcaccatcaccctgaccgccgacgtgatgagctacatccacagcatgaacagcagcatcctggaggactggaacttcggcgtgccccccccccccaccaccagcctggtggacacctaccggttcgtgcagagcgtggccatcacctgccagaaggacgccgcccccgccgagaacaaggacccctacgacaagctgaagttctggaacgtggacctgaaggagaagttcagcctggacctggaccagtaccccctgggccggaagttcctggtgcaggccggcctgcggcggaagcccaccatcggcccccggaagcggagcgcccccagcgccaccaccagcagcaagcccgccaagcgggtgcgggtgcgggcccggaagtga

SEQ ID NO: 25 - DNA, 477 bp, Synthetic construct
  source 1..477: mol_type = other DNA
atgcaccagaagcggaccgccatgttccaggacccccaggagcggccccggaagctgccccagctgtgcaccgagctgcagaccaccatccacgacatcatcctggagtgcgtgtactgcaagcagcagctgctgcggcgggaggtgtacgacttcgccttccgggacctgtgcatcgtgtaccgggacggcaacccctacgccgtgtgcgacaagtgcctgaagttctacagcaagatcagcgagtaccggcactactgctacagcctgtacggcaccaccctggagcagcagtacaacaagcccctgtgcgacctgctgatccggtgcatcaactgccagaagcccctgtgccccgaggagaagcagcggcacctggacaagaagcagcggttccacaacatccggggccggtggaccggccggtgcatgagctgctgccggagcagccggacccggcgggagacccagctgatg

SEQ ID NO: 26 - DNA, 1638 bp, Synthetic construct
  source 1..1638: mol_type = other DNA
atggcccggggcgccggcctggtgttcttcgtgggcgtgtgggtggtgagctgcctggccgccgccccccggaccagctggaagcgggtgaccagcggcgaggacgtggtgctgctgcccgcccccgccgagcggacccgggcccacaagctgctgtgggccgccgagcccctggacgcctgcggccccctgcggcccagctgggtggccctgtggcccccccggcgggtgctggagaccgtggtggacgccgcctgcatgcgggcccccgagcccctggccatcgcctacagcccccccttccccgccggcgacgagggcctgtacagcgagctggcctggcgggaccgggtggccgtggtgaacgagagcctggtgatctacggcgccctggagaccgacagcggcctgtacaccctgagcgtggtgggcctgagcgacgaggcccggcaggtggccagcgtggtgctggtggtggagcccgcccccgtgcccacccccacccccgacgactacgacgaggaggacgacgccggcgtgaccaacgcccggcggagcgccttccccccccagccccccccccggcggccccccgtggccccccccacccacccccgggtgatccccgaggtgagccacgtgcggggcgtgaccgtgcacatggagaccctggaggccatcctgttcgcccccggcgagaccttcggcaccaacgtgagcatccacgccatcgcccacgacgacggcccctacgccatggacgtggtgtggatgcggttcgacgtgcccagcagctgcgccgacatgcggatctacgaggcctgcctgtaccacccccagctgcccgagtgcctgagccccgccgacgccccctgcgccgtgagcagctgggcctaccggctggccgtgcggagctacgccggctgcagccggaccaccccccccccccggtgcttcgccgaggcccggatggagcccgtgcccggcctggcctggctggccagcaccgtgaacctggagttccagcacgccagcccccagcacgccggcctgtacctgtgcgtggtgtacgtggacgaccacatccacgcctggggccacatgaccatcagcaccgccgcccagtaccggaacgccgtggtggagcagcacctgccccagcggcagcccgagcccgtggagcccacccggccccacgtgcgggccccccaccccgcccccagcgcccggggccccctgcggctgggcgccgtgctgggcgccgccctgctgctggccgccctgggcctgagcgcctgggcctgcatgacctgctggcggcggcggagctggcgggccgtgaagagccgggccagcgccaccggccccacctacatccgggtggccgacagcgagctgtacgccgactggagcagcgacagcgagggcgagcgggacggcagcctgtggcaggacccccccgagcggcccgacagccccagcaccaacggcagcggcttcgagatcctgagccccaccgcccccagcgtgtacccccacagcgagggccggaagagccggcggcccctgaccaccttcggcagcggcagccccggccggcggcacagccaggccagctaccccagcgtgctgtggtga

SEQ ID NO: 27 - DNA, 1185 bp, Synthetic construct
  source 1..1185: mol_type = other DNA
atgggcggcgccgccgcccggctgggcgccgtgatcctgttcgtggtgatcgtgggcctgcacggcgtgcggagcaagtacgccctggtggacgccagcctgaagatggccgaccccaaccggttccggggcaaggacctgcccgtgctggaccagctgaccgacccccccggcgtgcggcgggtgtaccacatccaggccggcctgcccgaccccttccagccccccagcctgcccatcaccgtgtactacgccgtgctggagcgggcctgccggagcgtgctgctgaacgcccccagcgaggccccccagatcgtgcggggcgccagcgaggacgtgcggaagcagccctacaacctgaccatcgcctggttccggatgggcggcaactgcgccatccccatcaccgtgatggagtacaccgagtgcagctacaacaagagcctgggcgcctgccccatccggacccagccccggtggaactactacgacagcttcagcgccgtgagcgaggacaacctgggcttcctgatgcacgcccccgccttcgagaccgccggcacctacctgcggctggtgaagatcaacgactggaccgagatcacccagttcatcctggagcaccgggccaagggcagctgcaagtacgccctgcccctgcggatcccccccagcgcctgcctgagcccccaggcctaccagcagggcgtgaccgtggacagcatcggcatgctgccccggttcatccccgagaaccagcggaccgtggccgtgtacagcctgaagatcgccggctggcacggccccaaggccccctacaccagcaccctgctgccccccgagctgagcgagacccccaacgccacccagcccgagctggcccccgaggaccccgaggacagcgccctgctggaggaccccgtgggcaccgtggccccccagatcccccccaactggcacatccccagcatccaggacgccgccaccccctaccacccccccgccacccccaacaacatgggcctgatcgccggcgccgtgggcggcagcctgctggccgccctggtgatctgcggcatcgtgtactggatgcggcggcacacccagaaggcccccaagcggatccggctgccccacatccgggaggacgaccagcccagcagccaccagcccctgttctactag

SEQ ID NO: 28 - DNA, 1698 bp, Synthetic construct
  source 1..1698: mol_type = other DNA
atgaaggccaacctgctggtgctgctgtgcgccctggccgccgccgacgccgacaccatctgcatcggctaccacgccaacaacagcaccgacaccgtggacaccgtgctggagaagaacgtgaccgtgacccacagcgtgaacctgctggaggacagccacaacggcaagctgtgccggctgaagggcatcgcccccctgcagctgggcaagtgcaacatcgccggctggctgctgggcaaccccgagtgcgaccccctgctgcccgtgcggagctggagctacatcgtggagacccccaacagcgagaacggcatctgctaccccggcgacttcatcgactacgaggagctgcgggagcagctgagcagcgtgagcagcttcgagcggttcgagatcttccccaaggagagcagctggcccaaccacaacaccaacggcgtgaccgccgcctgcagccacgagggcaagagcagcttctaccggaacctgctgtggctgaccgagaaggagggcagctaccccaagctgaagaacagctacgtgaacaagaagggcaaggaggtgctggtgctgtggggcatccaccacccccccaacagcaaggagcagcagaacctgtaccagaacgagaacgcctacgtgagcgtggtgaccagcaactacaaccggcggttcacccccgagatcgccgagcggcccaaggtgcgggaccaggccggccggatgaactactactggaccctgctgaagcccggcgacaccatcatcttcgaggccaacggcaacctgatcgcccccatgtacgccttcgccctgagccggggcttcggcagcggcatcatcaccagcaacgccagcatgcacgagtgcaacaccaagtgccagacccccctgggcgccatcaacagcagcctgccctaccagaacatccaccccgtgaccatcggcgagtgccccaagtacgtgcggagcgccaagctgcggatggtgaccggcctgcggaacatccccagcatccagagccggggcctgttcggcgccatcgccggcttcatcgagggcggctggaccggcatgatcgacggctggtacggctaccaccaccagaacgagcagggcagcggctacgccgccgaccagaagagcacccagaacgccatcaacggcatcaccaacaaggtgaacaccgtgatcgagaagatgaacatccagttcaccgccgtgggcaaggagttcaacaagctggagaagcggatggagaacctgaacaagaaggtggacgacggcttcctggacatctggacctacaacgccgagctgctggtgctgctggagaacgagcggaccctggacttccacgacagcaacgtgaagaacctgtacgagaaggtgaagagccagctgaagaacaacgccaaggagatcggcaacggctgcttcgagttctaccacaagtgcgacaacgagtgcatggagagcgtgcggaacggcacctacgactaccccaagtacagcgaggagagcaagctgaaccgggagaaggtggacggcgtgaagctggagagcatgggcatctaccagatcctggccatctacagcaccgtggccagcagcctggtgctgctggtgagcctgggcgccatcagcttctggatgtgcagcaacggcagcctgcagtgccggatctgcatcatg

SEQ ID NO: 29 - DNA, 1503 bp, Synthetic construct
  source 1..1503: mol_type = other DNA
atgggcgcccgggccagcgtgctgagcggcggcaagctggacgcctgggagaagatccggctgcggcccggcggcaagaagaagtaccggctgaagcacctggtgtgggccagccgggagctggagcggttcgccctgaaccccggcctgctggagacccccgagggctgcctgcagatcatcgagcagatccagcccgccatcaagaccggcaccgaggagctgaagagcctgttcaacctggtggccgtgctgtactgcgtgcaccggaagatcgacgtgaaggacaccaaggaggccctggacaagatcgaggagatccagaacaagagccagcagaagacccagcaggccgccgccgacaaggagaaggacaacaaggtgagccagaactaccccatcgtgcagaacgcccagggccagatggtgcaccaggccatcagcccccggaccctgaacgcctgggtgaaggtggtggaggagaaggccttcagccccgaggtgatccccatgttcagcgccctgagcgagggcgccaccccccaggacctgaacgccatgctgaacaccgtgggcggccaccaggccgccatgcagatgctgaaggacaccatcaacgaggaggccgccgagtgggaccgggtgcaccccgtgcacgccggccccatcccccccggccagatgcgggagccccggggcagcgacatcgccggcaccaccagcaccctgcaggagcagatcgcctggatgaccggcaaccccgccatccccgtgggcgacatctacaagcggtggatcatcctgggcctgaacaagatcgtgcggatgtacagccccgtgagcatcctggacatcaagcagggccccaaggagcccttccgggactacgtggaccggttcttcaagaccctgcgggccgagcaggccacccaggacgtgaagaactggatgaccgagaccctgctggtgcagaacgccaaccccgactgcaagaccatcctgcgggccctgggccagggcgccagcatcgaggagatgatgaccgcctgccagggcgtgggcggccccagccacaaggcccgggtgctggccgaggccatgagccaggtgaccaacaccaacaccgccatcatgatgcagaagggcaacttcaagggccagcggaagttcgtgaagtgcttcaactgcggcaaggagggccacatcgcccggaactgccgggccccccggaagaagggctgctggaagtgcggccgggagggccaccagatgaaggactgcaccgagcggcaggccaacttcctgggcaagatctggcccagcagcaagggccggcccggcaacttcctgcagagccggcccgagcccaccgccccccccgccgagagcttcggcttcggcgaggagatgacccccagccccaagcaggagcagctgaaggacaaggagccccccctggccagcctgcggagcctgttcggcagcgaccccctgctgcagtga

SEQ ID NO: 30 - DNA, 2586 bp, Synthetic construct
  source 1..2586: mol_type = other DNA
atgcgggtgaaggagatccagatgagctggcccaacctgtggaagtggggcgtgctgatcctgggcctggtgatcatgtgcagcgccagcaacaagctgtgggtgaccgtgtactacggcgtgcccgtgtggcgggacgccgagaccaccctgttctgcgccagcgacgccaaggcccacgagaccgagaagcacaacgtgtgggccacccacgcctgcgtgcccaccgaccccaacccccaggagatctacctggccaacgtgaccgagaacttcaacatgtggaagaactacatggtggagcagatgcaggaggacgtgatcagcctgtgggaccagagcctgaagccctgcgtgaagctgacccccctgtgcgtgaccctgaactgcaccaacgagatcaacgtgaacgtgaccaacatgaacgccaccaccggcctgcccaccgtgggcaacatcaccgacgagatgaagaactgcagcttcaacgtgaccaccgagatccgggacaagaagcagaaggtgtacgccctgttctacaagctggacatcgtgcccatcgacaaccggaacaacagcaacaacagcgagtacatgctgatcaactgcaacaccagcgtgatcaagcaggcctgccccaaggtgagcttcgaccccatccccatccactactgcacccccgccggctacgtgatcctgaagtgcaacgacaagaagttcgacggcaccggcccctgcaagaacgtgagcagcgtgcagtgcacccacggcatcaagcccgtggtgagcacccagctgctgctgaacggcagcctggccgaggaggagatcatcatccggagcgagaacctgaccaacaacgccaagaccatcatcgtgcacctgaacgagagcgtggagatcaactgcacccggcccaacaacaacacccggaagggcacccacatcggccccggccaggtgttctaccggaccggcgagatcgtgggcgacatccggaaggccttctgcgagatcaacggcaccaagtggaacaacaccctggcccgggtggccaagaagctgaagaactacttcaacaagaccatcgagttccagccccccagcggcggcgacctggagatcaccatgcaccacttcaactgccggggcgagttcttctactgcaacaccagcaagctgttcaacggcacctacgagggcaacgacaccatggagaacaacggcaccaccatcatcatcccctgccggatcaagcagatcatcaacatgtggcagggcgccggccaggccatgtacgccccccccgtggagggcaagatcaactgcgtgagcaacatcaccggcatcctgctgacccgggacggcggcaacaacaacagcaccaccgagatcttccggcccggcggcggcaacatcaaggacaactggcggagcgagctgtacaagtacaaggtggtggagatcgagcccctgggcatcgcccccacccgggccgagcggaaggtggtgcagcgggagaagcgggccgtgggcatcggcgccatgatcttcggcttcctgggcgccgccggcagcaccatgggcgccgccagcatcaccctgaccgtgcaggcccggcagctgctgagcggcatcgtgcagcagcagagcaacctgctgcgggccatcgaggcccagcagcacctgctgcagctgaccgtgtggggcatcaagcagctgcaggcccgggtgctggccgtggagcggtacctgaaggaccagcggctgctgggcctgtggggctgcagcggcaagatcatctgcaccaccgccgtgccctggaacagcagctggagcaacaagagctacgaggagatctggaacaacatgacctggatcgagtgggagcgggagatcagcaactacaccggccagatctacgccatcctggccgagagccagaaccagcaggacaagaacgagaaggagctgctggagctggacaagtgggccagcctgtggaactggttcgacatcaccaactggctgtggtacatcaagatcttcatcatgatcgtgggcggcctgatcggcctgcggatcgtgttcgccgtgctgagcatcgtgaaccgggtgcggcagggctacagccccctgagcttccagaccctgacccaccaccagcgggagcccgaccggcccgagcggatcgaggagggcggcggcgagcaggaccgggaccggagcgtgcggctggtgagcggcttcctggccctggtgtgggacgacctgcggagcctgtgcctgttcagctaccaccggctgcgggacttcatcctgatcgccacccggaccgtggagctgctgggccacagcagcctgaagggcctgaagcggggctgggagggcctgaagtacctgggcaacctgctggtgtactggggccaggagctgaagatcagcgccatcagcctgctgaacgccaccgccatcgccgtgggcggctggaccgaccggctgatcgaggtgatccagcgggcctggcgggccatcctgcacatcccccggcggatccggcagggcgccgagcgggccctgctgtga

SEQ ID NO: 31 - DNA, 786 bp, Synthetic construct
  source 1..786: mol_type = other DNA
atgatcgagacctacaaccagaccagcccccggagcgccgccaccggcctgcccatcagcatgaagatcttcatgtacctgctgaccgtgttcctgatcacccagatgatcggcagcgccctgttcgccgtgtacctgcaccggcggctggacaagatcgaggacgagcggaacctgcacgaggacttcgtgttcatgaagaccatccagcggtgcaacaccggcgagcggagcctgagcctgctgaactgcgaggagatcaagagccagttcgagggcttcgtgaaggacatcatgctgaacaaggaggagaccaagaaggagaacagcttcgagatgcagaagggcgaccagaacccccagatcgccgcccacgtgatcagcgaggccagcagcaagaccaccagcgtgctgcagtgggccgagaagggctactacaccatgagcaacaacctggtgaccctggagaacggcaagcagctgaccgtgaagcggcagggcctgtactacatctacgcccaggtgaccttctgcagcaaccgggaggccagcagccaggcccccttcatcgccagcctgtgcctgaagagccccggccggttcgagcggatcctgctgcgggccgccaacacccacagcagcgccaagccctgcggccagcagagcatccacctgggcggcgtgttcgagctgcagcccggcgccagcgtgttcgtgaacgtgaccgaccccagccaggtgagccacggcaccggcttcaccagcttcggcctgctgaagctgtga

SEQ ID NO: 32 - DNA, 213 bp, Synthetic construct
  source 1..213: mol_type = other DNA
atgggcggccggcggcggaagaagcggaccaacgacaccaagcacgtgcggttcgccgccgccgtggaggtgtgggaggccgacgacatcgagcggaagggcccctgggagcaggtggccgtggaccggttccggttccagcggcggatcgccagcgtggaggagctgctgagcaccgtgctgctgcggcagaagaagctgctggagcagcag

SEQ ID NO: 33 - DNA, 1206 bp, Synthetic construct
  source 1..1206: mol_type = other DNA
atgatcatcatcgtgatcttcctgatgtgcctgaagatcgtgctgaacaacatcatcatctggagcaccctgaaccagaccgtgttcctgaacaacatcttcaccatcaacgacacctacggcggcctgttctggaacacctactacgacaacaaccggagcaacttcacctactgcggcatcgccggcaactactgcagctgctgcggccacaacatcagcctgtacaacaccaccaacaactgcagcctgatcatcttccccaacaacaccgagatcttcaaccggacctacgagctggtgtacctggacaagaagatcaactacaccgtgaagctgctgaagagcgtggacagccccaccatcacctacaactgcaccaacagcctgatcacctgcaagaacaacaacggcaccaacgtgaacatctacctgatcatcaacaacaccatcgtgaacgacaccaacggcgacatcctgaactactactggaacggcaacaacaacttcaccgccacctgcatgatcaacaacaccatcagcagcctgaacgagaccgagaacatcaactgcaccaaccccatcctgaagtaccagaactacctgagcaccctgttctacatcatcatcttcatcgtgagcggcctgatcatcggcatcttcatcagcatcatcagcgtgctgagcatccggcggaagcggaagaagcacgtggaggagatcgagagccccccccccagcgagagcaacgaggaggacatcagccacgacgacaccaccagcatccacgagcccagcccccgggagcccctgctgcccaagccctacagccggtaccagtacaacacccccatctactacatgcggcccagcacccagcccctgaaccccttccccctgcccaagccctgccccccccccaagccctgccccccccccaagccctgccccccccccaagccctgccccccccccaagccctgcagcccccccaagccctgccggccccccaagccctgccccccccccaagccctgccccccccccaagccctgccccccccccaagccctgcccccccagcaagccctgccccagccccgagagctacagcccccccaagcccctgcccagcatccccctgctgcccaacatcccccccctgagcacccagaacatcagcctgatccacgtggaccggatcatc

SEQ ID NO: 34 - DNA, 705 bp, Synthetic construct
  source 1..705: mol_type = other DNA
atgcggggcatcctgatcgccatcgagggcatcaacggcgtgggcaagagcacccaggccatccggctgaagaacgccctggagaacaagcggtacgacgtgatctacatgcacttccccagccccaacaccgacaccggcaagctgatcctggacgtgctgaacaagatcgtgaagatgcccagcgagcagctgcacgagctgttcaccaagcaccggtgcgagttcaccgccgagatcgccgccctgctgaagctgaacttcatcgtgatcgtggaccggtacatctggagcggcctggcctacgcccaggccgaccggatcatgatcgacaccaagaacaccttcaaccccgactacaccttcttcctgagcagcaacaagcccctgaacgagaagcccctgcacctgcagcggctgtacgagaccgaggagaagcaggagatcatcttcacccagttcatcaacatcatcaacgaggtgcccaaggacaagttctgcgccatccccgccaacctgaacaaggagatcatcgacaagatcatcttcagcaagaccatcaaggtgttcgagaagaacctgaacctggactacatcaagatgtacgacggcatgtacttcaacatccacgacctggacctgatccggttcgactggcagaagtgcatcgaggaggacgacggcatcgacgaggagtacggcctg

SEQ ID NO: 35 - DNA, 1771 bp, Synthetic construct
  source 1..1771: mol_type = other DNA
atggcccctgaacgtgagcttcaccaaccggaactacgacctggactacgacagcgtgcagccctacttctactgcgacgaggaggagaacttctaccagcagcagcagcagagcgagctgcagccccccgcccccagcgaggacatctggaagaagttcgagctgctgcccgccccccccctgagccccagccggcggagcggcctgtgcagccccagctacgtggccgtgacccccttcagcctgcggggcgacaacgacggcggcggcggcagcttcagcaccgccgaccagctggagatggtgaccgagctgctgggcggcgacatggtgaaccagagcttcatctgcgaccccgacgacgagaccttcatcaagaacatcatcatccaggactgcatgtggagcggcttcagcgccgccgccaagctggtgagcgagaagctggccagctaccaggccgcccggaaggacagcggcagccccaaccccgcccggggccacagcgtgtgcagcaccagcagcctgtacctgcaggacctgagcgccgccgccagcgagtgcatcgaccccagcgtggtgttcccctaccccctgaacgacagcagcagccccaagagctgcgccagccaggacagcagcgccttcagccccagcagcgacagcctgctgagcagcaccgagagcagcccccagggcagccccgagcccctggtgctgcacgaggagaccccccccaccaccagcagcgacagcgaggaggagcaggaggacgaggaggagatcgacgtggtgagcgtggagaagcggcaggcccccggcaagcggagcgagagcggcagccccagcgcctgctgcacctgctgcggcggcaccggcacctgcggcaccaccaccggcggcgccggcggcgccggcaccggcacctgcggcggcggcaccggcgcctgctgcgccggcggcgccggcaccacctgcacctgctgcgcctgcggcggcaccggctgcgccggcgccggcggcaccggcaccggcaccgccggcacctgcggcaccggcaccaccggcgccaccggctgcggcacctgcggctgcggcggcgccggcggcggcgccggcggcaccggcgccggctgctgcaccacctgctgcaccggcgccaccgccggcggcgcctgcggcgcctgcggcggcaccacctgcacctgctgctgcgccggcaccacctgcgccgcctgctgcaccggcacctgcgcctgcgccggcacctgcacctgcgccggcggcgcctgcacctgcaccggcggccacagcaagcccccccacagccccctggtgctgaagcggtgccacgtgagcacccaccagcacaactacgccgccccccccagcacccggaaggactaccccgccgccaagcgggtgaagctggacagcgtgcgggtgctgcggcagatcagcaacaaccggaagtgcaccagcccccggagcagcgacaccgaggagaacgtgaagcggcggacccacaacgtgctggagcggcagcggcggaacgagctgaagcggagcttcttcgccctgcgggaccagatccccgagctggagaacaacgagaaggcccccaaggtggtgatcctgaagaaggccaccgcctacatcctgagcgtgcaggccgaggagcagaagctgatcagcgaggaggacctgctgcggaagcggcgggagcagctgaagcacaagctggagcagctgcggaacagctgcgcctga

SEQ ID NO: 36 - DNA, 1644 bp, Synthetic construct
  source 1..1644: mol_type = other DNA
atggccgtgagcgacgccctgctgcccagcttcagcaccttcgccagcggccccgccggccgggagaagaccctgcggcaggccggcgcccccaacaaccggtggcgggaggagctgagccacatgaagcggctgccccccgtgctgcccggccggccctacgacctggccgccgccaccgtggccaccgacctggagagcggcggcgccggcgccgcctgcggcggcagcaacctggcccccctgccccggcgggagaccgaggagttcaacgacctgctggacctggacttcatcctgagcaacagcctgacccacccccccgagagcgtggccgccaccgtgagcagcagcgccagcgccagcagcagcagcagccccagcagcagcggccccgccagcgcccccagcacctgcagcttcacctaccccatccgggccggcaacgaccccggcgtggcccccggcggcaccggcggcggcctgctgtacggccgggagagcgcccccccccccaccgcccccttcaacctggccgacatcaacgacgtgagccccagcggcggcttcgtggccgagctgctgcggcccgagctggaccccgtgtacatccccccccagcagccccagccccccggcggcggcctgatgggcaagttcgtgctgaaggccagcctgagcgcccccggcagcgagtacggcagccccagcgtgatcagcgtgagcaagggcagccccgacggcagccaccccgtggtggtggccccctacaacggcggccccccccggacctgccccaagatcaagcaggaggccgtgagcagctgcacccacctgggcgccggcccccccctgagcaacggccaccggcccgccgcccacgacttccccctgggccggcagctgcccagccggaccacccccaccctgggcctggaggaggtgctgagcagccgggactgccaccccgccctgcccctgccccccggcttccacccccaccccggccccaactaccccagcttcctgcccgaccagatgcagccccaggtgccccccctgcactaccaggagctgatgccccccggcagctgcatgcccgaggagcccaagcccaagcggggccggcggagctggccccggaagcggaccggccccggcctgatgggccggaaggacggcctggtgtacgtgggcgtgcagggcggcgaggtgatggtgctggagtacggcggcccccggacctacggcctgctgggcttcggcttcagccccagcgccagcaccggcgccttcagctgggccacccacacctgcgactacgccggctgcggcaagacctacaccaagagcagccacctgaaggcccacctgcggacccacaccggcgagaagccctaccactgcgactgggacggctgcggctggaagttcgcccggagcgacgagctgacccggcactaccggaagcacaccggccaccggcccttccagtgccagaagtgcgaccgggccttcagccggagcgaccacctggccctgcacatgaagcggcacttctgaagcagcgtgcccttccccgagggcgaggccttcccccccgtgagcgtgaccaccctgggcagccccatgcacagcaactga

SEQ ID NO: 37 - DNA, 954 bp, Synthetic construct
  source 1..954: mol_type = other DNA
atgtacaacatgatggagaccgagctgaagccccccggcccccagcagaccagcggcggcggcggcggcaacagcaccgccgccgccgccggcggcaaccagaagaacagccccgaccgggtgaagcggcccatgaacgccttcatggtgtggagccggggccagcggcggaagatggcccaggagaaccccaagatgcacaacagcgagatcagcaagcggctgggcgccgagtggaagctgctgagcgagaccgagaagcggcccttcatcgacgaggccaagcggctgcgggccctgcacatgaaggagcaccccgactacaagtaccggccccggcggaagaccaagaccctgatgaagaaggacaagtacaccctgcccggcggcctgctggcccccggcggcaacagcatggccagcggcgtgggcgtgggcgccggcctgggcgccggcgtgaaccagcggatggacagctacgcccacatgaacggctggagcaacggcagctacagcatgatgcaggaccagctgggctacccccagcaccccggcctgaacgcccacggcgccgcccagatgcagcccatgcaccggtacgacgtgagcgccctgcagtacaacagcatgaccagcagccagacctacatgaacggcagccccacctacagcatgagctacagccagcagggcacccccggcatggccctgggcagcatgggcagcgtggtgaagagcgaggccagcagcagcccccccgtggtgaccagcagcagccacagccgggccccctgccaggccggcgacctgcgggacatgatcagcatgtacctgcccggcgccgaggtgcccgagcccgccgcccccagccggctgcacatgagccagcactaccagagcggccccgtgcccggcaccgccatcaacggcaccctgcccctgagccacatgtga

SEQ ID NO: 38 - DNA, 1083 bp, Synthetic construct
  source 1..1083: mol_type = other DNA
atggccggccacctggccagcgacttcgccttcagccccccccccggcggcggcggcgacggccccggcggccccgagcccggctgggtggacccccggacctggctgagcttccagggcccccccggcggccccggcatcggccccggcgtgggccccggcagcgaggtgtggggcatccccccctgcccccccccctacgagttctgcggcggcatggcctactgcggcccccaggtgggcgtgggcctggtgccccagggcggcctggagaccagccagcccgagggcgaggccggcgtgggcgtggagagcaacagcgacggcgccagccccgagccctgcaccgtgacccccggcgccgtgaagctggagaaggagaagctggagcagaaccccgaggagagccaggacatcaaggccctgcagaaggagctggagcagttcgccaagctgctgaagcagaagcggatcaccctgggctacacccaggccgacgtgggcctgaccctgggcgtgctgttcggcaaggtgttcagccagaccaccatctgccggttcgaggccctgcagctgagcttcaagaacatgtgcaagctgcggcccctgctgcagaagtgggtggaggaggccgacaacaacgagaacctgcaggagatctgcaaggccgagaccctggtgcaggcccggaagcggaagcggaccagcatcgagaaccgggtgcggggcaacctggagaacctgttcctgcagtgccccaagcccaccctgcagcagatcagccacatcgcccagcagctgggcctggagaaggacgtggtgcgggtgtggttctgcaaccggcggcagaagggcaagcggagcagcagcgactacgcccagcgggaggacttcgaggccgccggcagccccttcagcggcggccccgtgagcttccccctggcccccggcccccacttcggcacccccggctacggcagcccccacttcaccgccctgtacagcagcgtgcccttccccgagggcgaggccttcccccccgtgagcgtgaccaccctgggcagccccatgcacagcaactga

SEQ ID NO: 39 - DNA, 630 bp, Synthetic construct
  source 1..630: mol_type = other DNA
atgggcagcgtgagcaaccagcagttcgccggcggctgcgccaaggccgccgaggaggcccccgaggaggcccccgaggacgccgcccgggccgccgacgagccccagctgctgcacggcgccggcatctgcaagtggttcaacgtgcggatgggcttcggcttcctgagcatgaccgcccgggccggcgtggccctggacccccccgtggacgtgttcgtgcaccagagcaagctgcacatggagggcttccggagcctgaaggagggcgaggccgtggagttcaccttcaagaagagcgccaagggcctggagagcatccgggtgaccggccccggcggcgtgttctgcatcggcagcgagcggcggcccaagggcaagagcatgcagaagcggcggagcaagggcgaccggtgctacaactgcggcggcctggaccaccacgccaaggagtgcaagctgcccccccagcccaagaagtgccacttctgccagagcatcagccacatggtggccagctgccccctgaaggcccagcagggccccagcgcccagggcaagcccacctacttccgggaggaggaggaggagatccacagccccaccctgctgcccgaggcccagaactga

SEQ ID NO: 40 - DNA, 455 bp, Synthetic construct
  source 1..455: mol_type = other DNA
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgaagctcaaccatggggactgttttagagctagaaatagcaagttaaaataaggctagtccgttatcaacttgaaaaagtggcaccgagtcggtgctttttttctagacccagctttcttgtacaaagttggcatta

SEQ ID NO: 41 - DNA, 20 bp, Synthetic construct
  source 1..20: mol_type = other DNA
taagctaacctgtcacccca

SEQ ID NO: 42 - DNA, 20 bp, Synthetic construct
  source 1..20: mol_type = other DNA
agagcacagaggcgaggctt

SEQ ID NO: 43 - DNA, 24 bp, Synthetic construct
  source 1..24: mol_type = other DNA
ctcaaaagacttctgttcttgagc

SEQ ID NO: 44 - DNA, 24 bp, Synthetic construct
  source 1..24: mol_type = other DNA
cactgaacactgtgataatctgct

SEQ ID NO: 45 - DNA, 24 bp, Synthetic construct
  source 1..24: mol_type = other DNA
ccatggaattcgaacgctgacgtc

SEQ ID NO: 46 - DNA, 23 bp, Synthetic construct
  source 1..23: mol_type = other DNA
tatgggctatgaactaatgaccc

SEQ ID NO: 47 - DNA, 1584 bp, Synthetic construct
  source 1..1584: mol_type = other DNA
atggtgccccaggccctgctgttcgtgcccctgctggtgttccccctgtgcttcggcaagttccccatctacaccatccccgacaagctgggcccctggagccccatcgacatccaccacctgagctgccccaacaacctggtggtggaggacgagggctgcaccaacctgagcggcttcagctacatggagctgaaggtgggctacatcagcgccatcaagatgaacggcttcacctgcaccggcgtggtgaccgaggccgagacctacaccaacttcgtgggctacgtgaccaccaccttcaagcggaagcacttccggcccacccccgacgcctgccgggccgcctacaactggaagatggccggcgacccccggtacgaggagagcctgcacaacccctaccccgactaccactggctgcggaccgtgaagaccaccaaggagagcctggtgatcatcagccccagcgtggccgacctggacccctacgaccggagcctgcacagccgggtgttccccggcggcaactgcagcggcgtggccgtgagcagcacctactgcagcaccaaccacgactacaccatctggatgcccgagaacccccggctgggcatgagctgcgacatcttcaccaacagccggggcaagcgggccagcaagggcagcgagacctgcggcttcgtggacgagcggggcctgtacaagagcctgaagggcgcctgcaagctgaagctgtgcggcgtgctgggcctgcggctgatggacggcacctgggtggccatgcagaccagcaacgagaccaagtggtgcccccccggccagctggtgaacctgcacgacttccggagcgacgagatcgagcacctggtggtggaggagctggtgaagaagcgggaggagtgcctggacgccctggagagcatcatgaccaccaagagcgtgagcttccggcggctgagccacctgcggaagctggtgcccggcttcggcaaggcctacaccatcttcaacaagaccctgatggaggccgacgcccactacaagagcgtgcggacctggaacgagatcatccccagcaagggctgcctgcgggtgggcggccggtgccacccccacgtgaacggcgtgttcttcaacggcatcatcctgggccccgacggcaacgtgctgatccccgagatgcagagcagcctgctgcagcagcacatggagctgctggtgagcagcgtgatccccctgatgcaccccctggccgaccccagcaccgtgttcaagaacggcgacgaggccgaggacttcgtggaggtgcacctgcccgacgtgcacgagcggatcagcggcgtggacctgggcctgcccaactggggcaagtacgtgctgctgagcgccggcgccctgaccgccctgatgctgatcatcttcctgatgacctgctggcggcgggtgaaccggagcgagcccacccagcacaacctgcggggcaccggccgggaggtgagcgtgaccccccagagcggcaagatcatcagcagctgggagagctacaagagcggcggcgagaccggcctgtgaggcctgtga

SEQ ID NO: 48 - DNA, 3813 bp, Synthetic construct
  source 1..3813: mol_type = other DNA
atgttcgtgttcctggtgctgctgcccctggtgagcagccagtgcgtgaacctgcggacccggacccagctgccccccgcctacaccaacagcttcacccggggcgtgtactaccccgacaaggtgttccggagcagcgtgctgcacagcacccaggacctgttcctgcccttcttcagcaacgtgacctggttccacgccatccacgtgagcggcaccaacggcaccaagcggttcgacaaccccgtgctgcccttcaacgacggcgtgtacttcgccagcaccgagaagagcaacatcatccggggctggatcttcggcaccaccctggacagcaagacccagagcctgctgatcgtgaacaacgccaccaacgtggtgatcaaggtgtgcgagttccagttctgcaacgaccccttcctggacgtgtactaccacaagaacaacaagagctggatggagagcggcgtgtacagcagcgccaacaactgcaccttcgagtacgtgagccagcccttcctgatggacctggagggcaagcagggcaacttcaagaacctgcgggagttcgtgttcaagaacatcgacggctacttcaagatctacagcaagcacacccccatcaacctggtgcgggacctgccccagggcttcagcgccctggagcccctggtggacctgcccatcggcatcaacatcacccggttccagaccctgctggccctgcaccggagctacctgacccccggcgacagcagcagcggctggaccgccggcgccgccgcctactacgtgggctacctgcagccccggaccttcctgctgaagtacaacgagaacggcaccatcaccgacgccgtggactgcgccctggaccccctgagcgagaccaagtgcaccctgaagagcttcaccgtggagaagggcatctaccagaccagcaacttccgggtgcagcccaccgagagcatcgtgcggttccccaacatcaccaacctgtgccccttcggcgaggtgttcaacgccacccggttcgccagcgtgtacgcctggaaccggaagcggatcagcaactgcgtggccgactacagcgtgctgtacaacagcgccagcttcagcaccttcaagtgctacggcgtgagccccaccaagctgaacgacctgtgcttcaccaacgtgtacgccgacagcttcgtgatccggggcgacgaggtgcggcagatcgcccccggccagaccggcaagatcgccgactacaactacaagctgcccgacgacttcaccggctgcgtgatcgcctggaacagcaacaacctggacagcaaggtgggcggcaactacaactaccggtaccggctgttccggaagagcaacctgaagcccttcgagcgggacatcagcaccgagatctaccaggccggcagcaagccctgcaacggcgtggagggcttcaactgctacttccccctgcagagctacggcttccagcccaccaacggcgtgggctaccagccctaccgggtggtggtgctgagcttcgagctgctgcacgcccccgccaccgtgtgcggccccaagaagagcaccaacctggtgaagaacaagtgcgtgaacttcaacttcaacggcctgaccggcaccggcgtgctgaccgagagcaacaagaagttcctgcccttccagcagttcggccgggacatcgccgacaccaccgacgccgtgcgggacccccagaccctggagatcctggacatcaccccctgcagcttcggcggcgtgagcgtgatcacccccggcaccaacaccagcaaccaggtggccgtgctgtaccagggcgtgaactgcaccgaggtgcccgtggccatccacgccgaccagctgacccccacctggcgggtgtacagcaccggcagcaacgtgttccagacccgggccggctgcctgatcggcgccgagcacgtgaacaacagctacgagtgcgacatccccatcggcgccggcatctgcgccagctaccagacccagaccaacagccggcggcgggcccggagcgtggccagccagagcatcatcgcctacaccatgagcctgggcgccgagaacagcgtggcctacagcaacaacagcatcgccatccccaccaacttcaccatcagcgtgaccaccgagatcctgcccgtgagcatgaccaagaccagcgtggactgcaccatgtacatctgcggcgacagcaccgagtgcagcaacctgctgctgcagtacggcagcttctgcacccagctgaaccgggccctgaccggcatcgccgtggagcaggacaagaacacccaggaggtgttcgcccaggtgaagcagatctacaagaccccccccatcaaggacttcggcggcttcaacttcagccagatcctgcccgaccccagcaagcccagcaagcggagcttcatcgaggacctgctgttcaacaaggtgaccctggccgacgccggcttcatcaagcagtacggcgactgcctgggcgacatcgccgcccgggacctgatctgcgcccagaagttcaacggcctgaccgtgctgccccccctgctgaccgacgagatgatcgcccagtacaccagcgccctgctggccggcaccatcaccagcggctggaccttcggcgccggcgccgccctgcagatccccttcgccatgcagatggcctaccggttcaacggcatcggcgtgacccagaacgtgctgtacgagaaccagaagctgatcgccaaccagttcaacagcgccatcggcaagatccaggacagcctgagcagcaccgccagcgccctgggcaagctgcagaacgtggtgaaccagaacgcccaggccctgaacaccctggtgaagcagctgagcagcaacttcggcgccatcagcagcgtgctgaacgacatcctgagccggctggacccccccgaggccgaggtgcagatcgaccggctgatcaccggccggctgcagagcctgcagacctacgtgacccagcagctgatccgggccgccgagatccgggccagcgccaacctggccgccaccaagatgagcgagtgcgtgctgggccagagcaagcgggtggacttctgcggcaagggctaccacctgatgagcttcccccagagcgccccccacggcgtggtgttcctgcacgtgacctacgtgcccgcccaggagaagaacttcaccaccgcccccgccatctgccacgacggcaaggcccacttcccccgggagggcgtgttcgtgagcaacggcacccactggttcgtgacccagcggaacttctacgagccccagatcatcaccaccgacaacaccttcgtgagcggcaactgcgacgtggtgatcggcatcgtgaacaacaccgtgtacgaccccctgcagcccgagctggacagcttcaaggaggagctggacaagtacttcaagaaccacaccagccccgacgtggacctgggcgacatcagcggcatcaacgccagcgtggtgaacatccagaaggagatcgaccggctgaacgaggtggccaagaacctgaacgagagcctgatcgacctgcaggagctgggcaagtacgagcagtacatcaagtggccctggtacatctggctgggcttcatcgccggcctgatcgccatcgtgatggtgaccatcatgctgtgctgcatgaccagctgctgcagctgcctgaagggctgctgcagctgcggcagctgctgcaagttcgacgaggacgacagcgagcccgtgctgaagggcgtgaagctgcactacacc